(19) United States
(12) Patent Application Publication
(10) Pub. No.: US 2007/0004648 A1
(43) Pub. Date: Jan. 4, 2007

(54) Glucopyranosyl-substituted benzyl-benzene derivatives, medicaments containing such compounds, their use and process for their manufacture

(76) Inventors: Frank Himmelsbach, Mittelbiberach (DE); Matthias Eckhardt, Biberach (DE); Peter Eickelmann, Mittelbiberach (DE); Leo Thomas, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/477,230

(22) Filed: Jun. 29, 2006

(30) Foreign Application Priority Data
Jun. 29, 2005 (EP) 05 014 000

(51) Int. Cl. A61K 31/7034

(52) U.S. Cl. 514/25; 536/4.1

(57) ABSTRACT

Glucopyranosyloxy-substituted benzyl-benzene derivatives of the general formula I 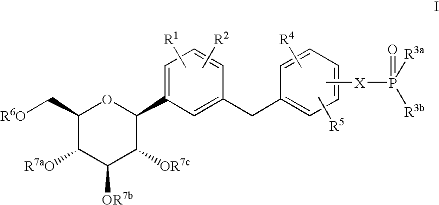
where the groups groups R¹, R², R^{3a}, R^{3b}, R⁴, R⁵, R⁶, X and R^{7a}, R^{7b}, R^{7c} are defined according to claim 1, including the tautomers, the stereoisomers thereof, the mixtures thereof and the salts thereof. The compounds according to the invention are suitable for the treatment of metabolic disorders.

This application claims priority benefit to EP 05 014 000.3, filed Jun. 29, 2005 the contents of which are incorporated herein.

The present invention relates to glucopyranosyl-substituted benzyl-benzene derivatives of the general formula I 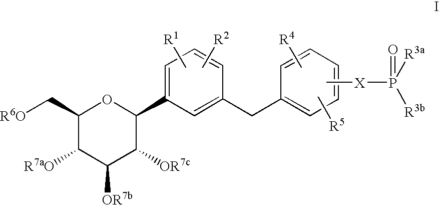
wherein the groups R¹, R², R^{3a}, R^{3b}, R⁴, R⁵, R⁶, X and R^{7a}, R^{7b}, R^{7c} are as defined hereinafter, including the tautomers, the stereoisomers, the mixtures thereof and the salts thereof. The invention further relates to pharmaceutical compositions containing a compound of formula I according to the invention as well as the use of a compound according to the invention for preparing a pharmaceutical composition for the treatment of metabolic disorders. In addition, the invention relates to processes for preparing a pharmaceutical composition as well as a compound according to the invention.

In the literature, compounds which have an inhibitory effect on the sodium-dependent glucose cotransporter SGLT2 are proposed for the treatment of diseases, particularly diabetes.

Glucopyranosyloxy-substituted aromatic groups and the preparation thereof and their possible activity as SGLT2 inhibitors are known from published International applications WO 98/31697, WO 01/27128, WO 02/083066, WO 03/099836, WO 2004/063209, WO 2004/080990, WO 2004/013118, WO 2004/052902, WO 2004/052903 and U.S. application Ser. No. US 2003/0114390.

### AIM OF THE INVENTION

The aim of the present invention is to find new pyranosyloxy-substituted benzene derivatives, particularly those which are active with regard to the sodium-dependent glucose cotransporter SGLT, particularly SGLT2. A further aim of the present invention is to discover pyranosyloxy-substituted benzene derivatives which have a good to very good inhibitory effect on the sodium-dependent glucose cotransporter SGLT2 in vitro and/or in vivo and/or have good to very good pharmacological and/or pharmacokinetic and/or physicochemical properties.

A further aim of the present invention is to provide new pharmaceutical compositions which are suitable for the prevention and/or treatment of metabolic disorders, particularly diabetes.

The invention also sets out to provide a process for preparing the compounds according to the invention.

Other aims of the present invention will become apparent to the skilled man directly from the foregoing and following remarks.

### OBJECT OF THE INVENTION

In a first aspect the present invention relates to glucopyranosyloxy-substituted benzyl-benzene derivatives of general formula I 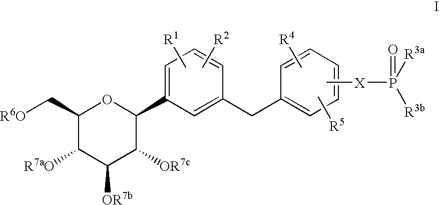
wherein
R¹ denotes hydrogen, fluorine, chlorine, bromine, iodine, C₁₋₄-alkyl, C₂₋₆-alkynyl, C₁₋₄- alkoxy, C₂₋₄-alkenyl-C₁₋₄-alkoxy, C₂₋₄-alkynyl-C₁₄-alkoxy, methyl substituted by 1 to 3 fluorine atoms, ethyl substituted by 1 to 5 fluorine atoms, methoxy substituted by 1 to 3 fluorine atoms, ethoxy substituted by 1 to 5 fluorine atoms, C₁₋₄-alkyl substituted by a hydroxy or C₁₋₃-alkoxy group, C₂₋₄-alkoxy substituted by a hydroxy or C₁₋₃-alkoxy group, C₂₋₆-alkenyl, C₃₋₇-cycloalkyl, C₃₋₇-cycloalkyl-C₁₋₃alkyl, C₃₋₇-cycloalkyloxy, C₃₋₇-cycloalkyl-C₁₋₃-alkoxy, C₅₋₇-cycloalkenyloxy, hydroxy, amino, nitro or cyano, while in the C₅₋₆-cycloalkyl groups a methylene group may be replaced by O;
R² denotes hydrogen, fluorine, chlorine, bromine, hydroxy, C₁₋₄-alkyl, C₁₋₄-alkoxy, cyano or nitro, while the alkyl or alkoxy group may be mono- or polysubstituted by fluorine, and
R^{3a}, R^{3b} independently of one another denote C₁₋₆-alkyl, C₂₋₆-alkenyl, C₂₋₆-alkynyl, C₃₋₇-cycloalkyl, C₃₋₇-cycloalkyl-C₁₋₃-alkyl, aryl, heteroaryl, aryl-C₁₋₃-alkyl, heteroaryl-C₁₋₃-alkyl, C₁₋₆-alkyloxy, C₄₋₇-cycloalkyloxy, hydroxy;
   wherein each C₁₋₆-alkyl group may be substituted with one to three substituents L2; and
   wherein aryl-groups may be substituted with one to three substituents L1; or
   R^{3a} and R^{3b} are linked together to form a C₄₋₅-alkylene, C₄₋₅-alkenylene, -O-C₃₋₄-alkylene, -O-C₂₋₃-alkylene-O- or -CH₂CH₂-O-CH₂CH₂-chain;
      wherein the alkylene moieties may be substituted with one to three substituents L2; and
      wherein two adjacent carbon atoms may be part of a further annelated 5- or 6-membered saturated or partially or fully unsaturated carbocyclic ring that may be additionally substituted with up to four substituents L1; and
R⁴, R⁵ independently of one another denote hydrogen, fluorine, chlorine, bromine, iodine, cyano, nitro, C₁₋₃-alkyl, C₁₋₃-alkoxy, or a methyl- or methoxy-group substituted by 1 to 3 fluorine atoms,
X denotes bond, O, C₁₋₅-alkylene, -O-CH₂CH₂-O, -O-CH₂CH₂-O-CH₂-, -CH₂-O-CH₂CH₂-O-, or an C₂₋₅-alkylene wherein one methylene unit is replaced by O;
   wherein the alkylene moieties may be substituted with one to three substituents L2,
L1 independently of one another are selected from among fluorine, chlorine, bromine, iodine, hydroxy, cyano, C₁₋₃-alkyl, difluoromethyl, trifluoromethyl, C₁₋₃-alkoxy, difluoromethoxy, trifluoromethoxy, amino, C₁₋₃-alkyl-amino and di(C₁₋₃-alkyl)-amino; and
L2 independently of one another are selected from among fluorine, hydroxyl, C₁₋₃-alkyl, difluoromethyl, trifluoromethyl, C₁₋₃-alkoxy, difluoromethoxy, trifluoromethoxy, cyano, amino, C₁₋₃-alkyl-amino and di(C₁₋₃-alkyl)-amino; and
R⁶, R^{7a},
R^{7b}, R^{7c} independently of one another have a meaning selected from among hydrogen, (C₁₋₁₈-alkyl)carbonyl, (C₁₋₁₈-alkyl)oxycarbonyl, arylcarbonyl and aryl-(C₁₋₃-alkyl)-carbonyl, while the aryl-groups may be mono- or disubstituted independently of one another by identical or different groups L1;

while by the aryl groups mentioned in the definition of the above groups are meant phenyl or naphthyl groups which may be substituted as defined; and

while by the heteroaryl groups mentioned in the definition of the above groups are meant a pyrrolyl, furanyl, thienyl, pyridyl, indolyl, benzofuranyl, benzothiophenyl, quinolinyl, isoquinolinyl or tetrazolyl group,

or is meant a pyrrolyl, furanyl, thienyl or pyridyl group, wherein one or two methyne groups are replaced by nitrogen atoms,

or is meant an indolyl, benzofuranyl, benzothiophenyl, quinolinyl or isoquinolinyl group, wherein one to three methyne groups are replaced by nitrogen atoms,

while the above-mentioned heteroaryl groups independently of one another may be mono- or disubstituted by identical or different groups L1;

while, unless otherwise stated, the above-mentioned alkyl groups may be straight or branched chain,

the tautomers, the stereoisomers thereof, the mixtures thereof and the salts thereof.

The compounds of general formula I according to the invention and the physiologically acceptable salts thereof have valuable pharmacological properties, particularly an inhibitory effect on the sodium-dependent glucose cotransporter SGLT, particularly SGLT2. Moreover compounds according to the invention may have an inhibitory effect on the sodium-dependent glucose cotransporter SGLT1. Compared with a possible inhibitory effect on SGLT1 the compounds according to the invention preferably inhibit SGLT2 selectively.

The present invention also relates to the physiologically acceptable salts of the compounds according to the invention with inorganic or organic acids.

This invention also relates to pharmaceutical compositions, containing at least one compound according to the invention or a physiologically acceptable salt according to the invention, optionally together with one or more inert carriers and/or diluents.

This invention also relates to the use of at least one compound according to the invention or one of the physiologically acceptable salts thereof for preparing a pharmaceutical composition which is suitable for the treatment or prevention or diseases or conditions which can be influenced by inhibiting the sodium-dependent glucose cotransporter SGLT, particularly SGLT2.

This invention also relates to the use of at least one compound according to the invention or one of the physiologically acceptable salts thereof for preparing a pharmaceutical composition which is suitable for the treatment of metabolic disorders.

This invention also relates to the use of at least one compound according to the invention or one of the physiologically acceptable salts thereof for preparing a pharmaceutical composition for inhibiting the sodium-dependent glucose cotransporter SGLT, particularly SGLT2.

The invention further relates to a process for preparing a pharmaceutical composition according to the invention, characterised in that a compound according to the invention or one of the physiologically acceptable salts thereof is incorporated in one or more inert carriers and/or diluents by a non-chemical method.

The present invention also relates to a process for preparing the compounds of general formula I according to the invention, characterised in that

a) in order to prepare compounds of general formula I which are defined as hereinbefore and hereinafter,

a compound of general formula II 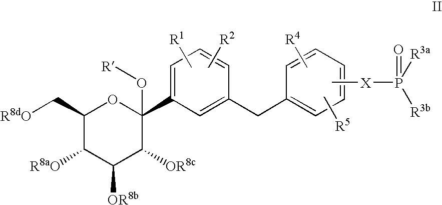
wherein
R¹ denotes H, C₁₋₄-alkyl, (C₁₋₈-alkyl)carbonyl, (C₁₋₁₈-alkyl)oxycarbonyl, arylcarbonyl and aryl-(C₁₋₃-alkyl)-carbonyl, wherein the alkyl or aryl groups may be mono- or polysubstituted by halogen;
R^{8a}, R^{8b}
R^{8c}, R^{8d} independently of one another have one of the meanings given hereinbefore and hereinafter for the groups R⁶, R^{7a}, R^{7b}, R^{7c}, or denote a benzyl group or a R^{a}R^{b}R^{c}Si group or a ketal or acetal group, particularly an alkylidene or arylalkylidene ketal or acetal group, while in each case two adjacent groups R^{8a}, R^{8b}, R^{8c}, R^{8d} may form a cyclic ketal or acetal group or a 1,2-di(C₁₋₃-alkoxy)-1,2-di(C₁₋₃-alkyl)-ethylene bridge, while the above-mentioned ethylene bridge forms, together with two oxygen atoms and the two associated carbon atoms of the pyranose ring, a substituted dioxane ring, particularly a 2,3-dimethyl-2,3-di(C₁₋₃-alkoxy)-1,4-dioxane ring, and while alkyl, aryl and/or benzyl groups may be mono- or polysubstituted by halogen or C₁₋₃-alkoxy, and while benzyl groups may also be substituted by a di-(C₁₋₃-alkyl)amino group; and
R^{a}, R^{b}, R^{c} independently of one another denote C₁₋₄-alkyl, aryl or aryl-C₁₋₃-alkyl, wherein the aryl or alkyl groups may be mono- or polysubstituted by halogen;

while by the aryl groups mentioned in the definition of the above groups are meant phenyl or naphthyl groups, preferably phenyl groups;

and wherein the groups X, R¹, R², R^{3a}, R^{3b}, R⁴, R⁵ and R⁶, R^{7a}, R^{7b}, R^{7c} are defined as hereinbefore and hereinafter;

is reacted with a reducing agent in the presence of a Lewis or Brønsted acid, while any protective groups present are cleaved simultaneously or subsequently; or

b) in order to prepare compounds of general formula I wherein R⁶, R7a, R^{7b} and R^{7c} denote hydrogen,

a compound of general formula III 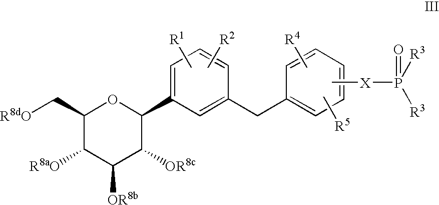
wherein R^{8a}, R^{8b}, R^{8c}, R^{8d}, X and R¹, R², R^{3a}, R^{3b}, R⁴, R⁵ are defined as hereinbefore and hereinafter, but at least one of the groups R^{8a}, R^{8b}, R^{8c}, R^{8d} does not denote hydrogen, is hydrolysed, and

if desired a compound of general formula I thus obtained wherein R⁶ denotes a hydrogen atom, is converted by acylation into a corresponding acyl compound of general formula I, and/or

if necessary any protective group used in the reactions described above is cleaved and/or

if desired a compound of general formula I thus obtained is resolved into its stereoisomers and/or

if desired a compound of general formula I thus obtained is converted into the salts thereof, particularly for pharmaceutical use into the physiologically acceptable salts thereof.

This invention further relates to a process for preparing compounds of general formula II′ 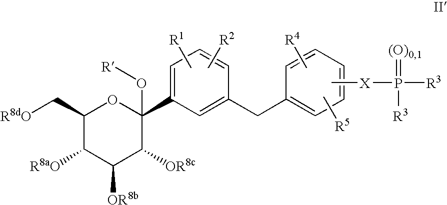
wherein
R¹ denotes H, C₁₋₄-alkyl, (C₁₋₁₈-alkyl)carbonyl, (C₁₋₁₈-alkyl)oxycarbonyl, arylcarbonyl and aryl-(C₁₋₃-alkyl)-carbonyl, wherein the alkyl or aryl groups may be mono- or polysubstituted by halogen;
R^{8a}, R^{8b},
R^{8c}, R^{8d} independently of one another have one of the meanings given for the groups R⁶ , R^{7a}, R^{7b}, R^{7c}, denote a benzyl group or a R^{a}R^{b}R^{c}Si group or a ketal or acetal group, while in each case two adjacent groups R^{8a}, R^{8b}, R^{8c}, R^{8d} may form a cyclic ketal or acetal group or may form, with two oxygen atoms of the pyranose ring, a substituted 2,3-oxydioxane ring, particularly a 2,3-dimethyl-2,3-di(C₁₋₃-alkoxy)-1,4-dioxane ring, and while alkyl, aryl and/or benzyl groups may be mono- or polysubstituted by halogen or C₁₋₃-alkoxy, and while benzyl groups may also be substituted by a di-(C₁₋₃-alkyl)amino group; and
R^{a}, R^{b}, R^{c} independently of one another denote C₁₋₄-alkyl, aryl or aryl-C₁₋₃-alkyl, while the alkyl or aryl groups may be mono- or polysubstituted by halogen;

while by the aryl groups mentioned in the definition of the above groups are meant phenyl or naphthyl groups, preferably phenyl groups;

and X R¹R², R^{3a}, R^{3b}, R⁴, R⁵, R⁶, R^{7a}, R^{7b}, R^{7c} are defined as hereinbefore and hereinafter,

wherein an organometallic compound (V) which may be obtained by halogen-metal exchange or by inserting a metal in the carbon-halogen bond of a halogen-benzylbenzene compound of general formula IVa or IVb 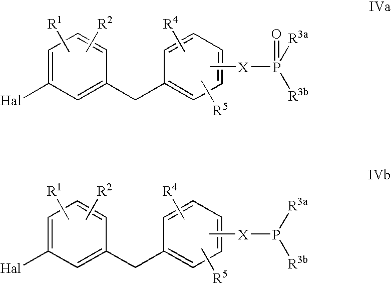
wherein Hal denotes Cl, Br and I and X, R¹, R², R^{3a}, R^{3b}, R⁴, R⁵ are defined as hereinbefore and hereinafter, and optionally subsequent transmetallation,

is added to a gluconolactone of general formula VI 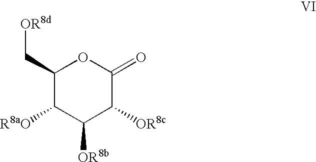
wherein R^{8a}, R^{8b}, R^{8c}, R^{8d} are defined as hereinbefore and hereinafter,
and

then the resulting adduct, is reacted, preferably in situ, with water or an alcohol R¹-OH, while R¹ denotes optionally substituted C₁₋₄-alkyl, in the presence of an acid, such as for example methanesulphonic acid, sulphuric acid, hydrochloric acid, acetic acid or ammonium chloride, and optionally the product obtained in the reaction with water wherein R¹ denotes H is converted, in a subsequent reaction, with an acylating agent, such as for example the corresponding acid chloride or anhydride, into the product of formula II wherein R¹ denotes (C₁₋₁₈-alkyl)carbonyl, (C₁₋₁₈-alkyl)oxycarbonyl, arylcarbonyl or aryl-(C₁₋₃-alkyl)-carbonyl, which may be substituted as specified. The intermediate products listed, particularly those of formula IVa and IVb, formula II and formula III, are also a subject of this invention.

### DETAILED DESCRIPTION OF THE INVENTION

Unless otherwise stated, the groups, residues and substituents, particularly R¹, R², R^{3a}, R^{3b}, R⁴, R⁵, X, L1, L2, R^{N}, R⁶, R^{7a}, R^{7b}, R^{7c}, R^{8a}, R^{8b}, R^{8c}, R^{8d}, are defined as above and hereinafter.

If residues, substituents or groups occur several times in a compound, as for example L1 and/or L2, they may have the same or different meanings.

Some preferred meanings of individual groups and substituents of the compounds according to the invention will be given hereinafter.

The group -X-P(O)R^{3a}R^{3b} is preferably in the meta or para position to the -CH₂-bridge, so that compounds according to the following formulae I.1 and I.2, particularly formula I.2, are preferred:

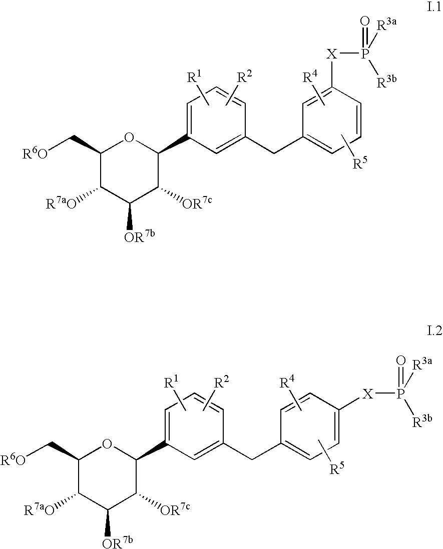

The group R¹ preferably denotes hydrogen, fluorine, chlorine, bromine, C₁₋₄-alkyl, C₁₋₄-alkoxy, methyl substituted by 1 to 3 fluorine atoms, methoxy substituted by 1 to 3 fluorine atoms, C₃₋₇-cycloalkyloxy or C₃₋₇-cycloalkyl-C₁₋₃-alkoxy, while in the C₅₋₆-cycloalkyl groups a methylene group may be replaced by O.

Particularly preferred meanings of R¹ are hydrogen, fluorine, chlorine, methyl, methoxy, ethoxy, cyclopentyloxy, cyclohexyloxy, tetrahydrofuran-3-yloxy and tetrahydropyran4-yl-oxy; particularly methyl and chlorine.

Preferred meanings of the group R² are hydrogen, fluorine, chlorine, methyl, methoxy, ethoxy and methyl substituted by 1 to 3 fluorine atoms.

Particularly preferred meanings of the group R² are hydrogen, fluorine, methoxy, ethoxy and methyl, particularly hydrogen.

The groups R^{3a}, R^{3b} preferably denote independently of one another C₁₋₄-alkyl, aryl, and C₃₋₇-cycloalkyl, wherein the alkylene parts may be substituted with one to three substituents L2 and wherein aryl-groups may be substituted with one to three substituents L1.

Particularly preferred meanings of R^{3a}, R^{3b} are independently of one another C₁₋₄-alkyl; particularly methyl, ethyl, propyl and isopropyl.

If the groups R^{3a}, R^{3b} are linked together they preferably form a C₄₋₅-alkylene chain wherein the alkylene chain may be substituted with one to three substituents L2; thus forming together with the phosphorous atom a 5- or 6-membered ring. A particularly preferred meaning of R3a, R^{3b} linked together according to this definition is a butylene group; thus forming together with the phosphorous atom a 1-oxophospholane group.

Preferred meanings of the group X are bond, C₁₋₅-alkylene, C₂₋₅-alkylene wherein one methylene unit is replaced by O and wherein the alkylene parts of the aforementioned groups may be substituted with one to three substituents L2 with the proviso that the phosphorous atom is attached to a carbon atom of the group X.

Particularly preferred meanings of the group X are bond, C₁₋₃-alkylene, O-CH₂, O-CH₂CH₂, CH₂O-CH₂ wherein the alkylene parts may be substituted with one to three substituents L2; particularly bond, methylene, ethylene, and -O-CH_{2.}

Preferred meanings of the group L1 independently of one another are selected from among fluorine, chlorine, bromine, cyano, hydroxy, C₁₋₃-alkyl, difluoromethyl, trifluoromethyl, C₁₋₃-alkoxy, difluoromethoxy, trifluoromethoxy and di(C₁₋₃-alkyl)-amino.

Particularly preferred meanings of the group L1 independently of one another are selected from among fluorine, chlorine, hydroxy, methyl, trifluoromethyl, ethyl, methoxy, ethoxy and dimethylamino, particularly methyl, ethyl, methoxy, ethoxy and dimethylamino.

Preferred meanings of the group L2 independently of one another are selected from among cyano, hydroxy, C₁₋₃-alkyl, difluoromethyl, trifluoromethyl, C₁₋₃-alkoxy, and di(C₁₋₃-alkyl)-amino.

Particularly preferred meanings of the group L2 are selected from fluorine, hydroxy, methyl, trifluoromethyl, ethyl, methoxy, ethoxy and dimethylamino, particularly methyl, ethyl, methoxy, ethoxy and dimethylamino.

Preferred meanings of the group R⁴ are hydrogen and fluorine, particularly hydrogen.

Preferred meanings of the group R⁵ are hydrogen and fluorine, particularly hydrogen.

The group R⁶ preferably denotes according to the invention hydrogen, (C₁₋₈-alkyl)oxycarbonyl, C₁₋₈-alkylcarbonyl or benzoyl, particularly hydrogen or (C₁₋₆-alkyl)oxycarbonyl or C₁₋₆-alkylcarbonyl, particularly preferably hydrogen, methylcarbonyl, methoxycarbonyl or ethoxycarbonyl, most particularly preferably hydrogen.

The substituents R^{7a}, R^{7b}, R^{7c} preferably represent independently of one another hydrogen, (C₁₋₈-alkyl)oxycarbonyl, (C₁₋₁₈-alkyl)carbonyl or benzoyl, particularly hydrogen, (C₁₋₆-alkyl)oxycarbonyl or (C₁₋₈-alkyl)carbonyl, particularly preferably hydrogen, methoxycarbonyl, ethoxycarbonyl, methylcarbonyl or ethylcarbonyl. Most particularly preferably R^{7a}, R^{7b} and R^{7c} represent hydrogen.

The compounds of formula I wherein R⁶, R^{7a}, R^{7b} and R^{7c} according to the invention have a meaning other than hydrogen, for example C₁₋₁₈-alkylcarbonyl, are preferably suitable as intermediate products for the synthesis of compounds of formula I wherein R⁶, R^{7a}, R^{7b} and R^{7c} denote hydrogen.

Particularly preferred compounds of general formula I are selected from among formulae I.2a to I.2d, particularly I.2c: 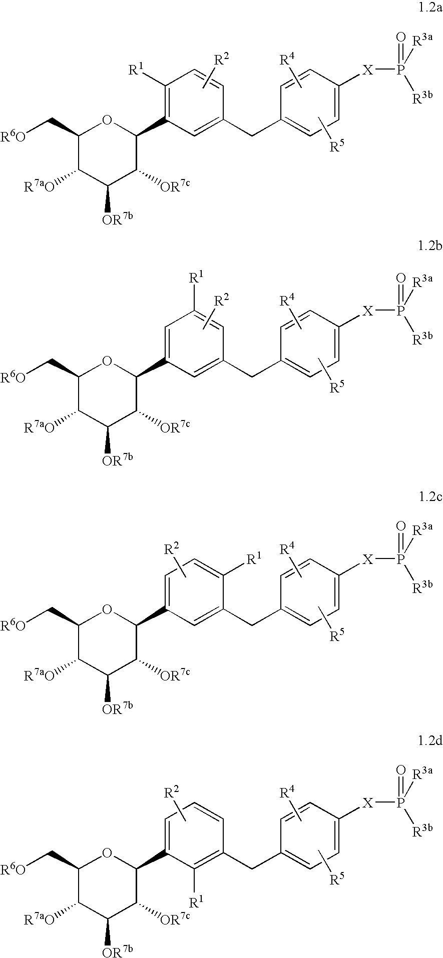
while the groups R¹, R², R^{3a}, R^{3b}, R⁴, R ⁵, R⁶, R^{7a}, R^{7b}, R^{7c} and X have one of the meanings given previously, particularly have one of the given meanings specified as being preferred; and particularly
R¹ denotes hydrogen, fluorine, chlorine, bromine, C₁₋₄-alkyl, C₁4-alkoxy, methyl substituted by 1 to 3 fluorine atoms, methoxy substituted by 1 to 3 fluorine atoms, C₃₋₇-cycloalkyloxy or C₃₋₇-cycloalkyl-C₁₋₃-alkoxy, while in the C₅₋₆-cycloalkyl groups a methylene group may be replaced by O; R¹ particularly preferably denotes hydrogen, fluorine, chlorine, methyl, methoxy, ethoxy, cyclopentyloxy, cyclohexyloxy, tetrahydrofuran-3-yloxy or tetrahydropyran4-yl-oxy; and
R² denotes hydrogen, fluorine, methoxy, ethoxy or methyl, particularly hydrogen; and
R^{3a}, R^{3b} independently of one another denote C₁₋₄-alkyl, phenyl, and C₃₋₇-cycloalkyl, wherein each C₁₋₆-alkyl group may be substituted with one to three substituents L2 and the phenyl group may be substituted with one to three substituents L1; particularly methyl, ethyl, n-propyl, i-propyl and -CH₂-CF₃; or
   R^{3a} and R^{3b} are linked together to form an C₄₋₅-alkylene chain wherein the alkylene chain may be substituted with one to three substituents L2; particularly to form a butylene chain;
R⁴ denotes hydrogen or fluorine, particularly hydrogen; and
R⁵ denotes hydrogen or fluorine, particularly hydrogen; and
X denotes bond, C₁₋₅-alkylene, C₂₋₅-alkylene wherein one methylene unit is replaced by O and wherein the alkylene parts of the aforementioned groups may be substituted with one to three substituents L2 with the proviso that the phosphorous atom is attached to a carbon atom of the group X; particularly X denotes bond, methylene, ethylene, propylene, -O-CH₂- and -CH₂-O-CH₂-;
L1 independently of one another are selected from among fluorine, chlorine, bromine, cyano, hydroxy, C₁₋₃-alkyl, difluoromethyl, trifluoromethyl, C₁₋₃-alkoxy, difluoromethoxy, trifluoromethoxy and di(C₁₋₃-alkyl)-amino; particularly selected from among fluorine, chlorine, hydroxy, methyl, trifluoromethyl, ethyl, methoxy, ethoxy and dimethylamino; most preferably selected from among methyl, ethyl, methoxy, ethoxy and dimethylamino; and
L2 independently of one another are selected from among fluorine, hydroxy, C₁₋₃-alkyl, difluoromethyl, trifluoromethyl, C₁₋₃-alkoxy, difluoromethoxy, trifluoromethoxy and di(C₁₋₃-alkyl)-amino; particularly selected from among fluorine, hydroxy, methyl, trifluoromethyl, ethyl, methoxy, ethoxy and dimethylamino; most preferably selected from among fluorine, methyl, ethyl, methoxy, ethoxy and dimethylamino; and;
R⁶ denotes hydrogen, (C₁₋₆-alkyl)oxycarbonyl, (C₁₋₆alkyl)carbonyl or benzoyl, particularly hydrogen, methylcarbonyl, methoxycarbonyl or ethoxycarbonyl, most particularly preferably hydrogen; and
R^{7a}, R^{7b}, R^{7c} independently of one another represent hydrogen, (C₁₋₆-alkyl)oxycarbonyl, (C₁₋₈-alkyl)carbonyl or benzoyl, particularly hydrogen, methoxycarbonyl, ethoxycarbonyl, methylcarbonyl or ethylcarbonyl, particularly preferably hydrogen;
   including the tautomers, the stereoisomers, the mixtures thereof and the salts thereof.

According to a variant of the embodiments given hereinbefore, other preferred compounds are those wherein the phenyl group which carries the substituent -X-P(O)R^{3a}R^{3b} has at least one other substituent R⁴ and/or R⁵ which is different from hydrogen. According to this variant, particularly preferred compounds are those which have a substituent R⁴ representing fluorine.

The compounds of general formula I specified in the experimental section that follows, and the derivatives thereof, wherein R⁶ has a meaning according to the invention other than hydrogen, particularly wherein R⁶ denotes ethoxycarbonyl or methoxycarbonyl, including the tautomers, the stereoisomers thereof and the mixtures thereof, are preferred according to another variant of this invention.

In the processes according to the invention the groups R¹, R², R^{3a}, R^{3b}, R⁴, R⁵, and X preferably have the meanings specified hereinbefore as being preferred. Moreover R¹ preferably denotes H, C₁₋₃-alkyl or benzyl, particularly H, ethyl or methyl. The groups R^{8a}, R^{8b}, R^{8c} and R^{8d} independently of one another preferably denote H, C₁₋₄-alkylcarbonyl or benzyl, particularly H, methylcarbonyl, ethylcarbonyl or benzyl.

The invention also relates to compounds of general formula IVa and IVb 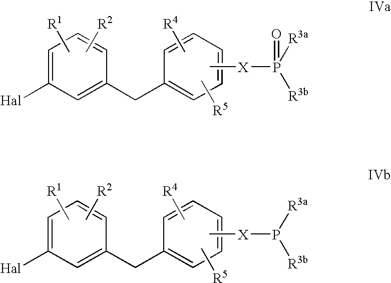
wherein Hal denotes chlorine, bromine or iodine and the groups X, R¹, R², R3a, R^{3b}, R⁴ and R⁵ are as hereinbefore defined, as intermediate products or starting materials in the synthesis of the compounds according to the invention. Particularly preferably, the groups X, R¹, R², R^{3a}, R^{3b}, R⁴ and R⁵ have the meanings given following formulae I.2a to I.2d.

The invention also relates to compounds of general formula II, particularly of general formula II.2 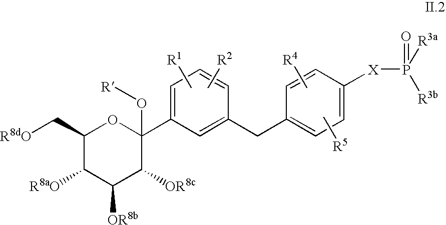
wherein R¹ , R^{8a}, R^{8b}, R^{8c}, R^{8d}, X, R¹, R², R^{3a}, R^{3b}, R⁴ and R⁵ are defined as hereinbefore and hereinafter; particularly wherein R¹ denotes H, C₁₋₃-alkyl or benzyl, particularly H, ethyl or methyl; and the groups R^{8a}, R^{8b}, R^{8c} and R^{8d} independently of one another represent H, C₁₋₄-alkylcarbonyl or benzyl, particularly H, methylcarbonyl, ethylcarbonyl or benzyl and the groups R¹, R², R^{3a}, R^{3b}, R⁴, R⁵, and X are as hereinbefore defined, as intermediate products or starting materials in the synthesis of the compounds according to the invention. Particularly preferably the groups R¹, R², R^{3a}, R^{3b}, R⁴, R⁵, and X have the meanings given following formulae I.2a to I.2d.

Some terms used above and hereinafter to describe the compounds according to the invention will now be defined more closely.

The term halogen denotes an atom selected from the group consisting of F, Cl, Br and I.

The term C₁₋ₙ-alkyl, wherein n may have a value of 1 to 18, denotes a saturated, branched or unbranched hydrocarbon group with 1 to n C atoms. Examples of such groups include methyl, ethyl, n-propyl, iso-propyl, butyl, iso-butyl, sec-butyl, tert-butyl, n-pentyl, iso-pentyl, neo-pentyl, tert-pentyl, n-hexyl, iso-hexyl, etc.

The term C₂₋ₙ-alkynyl, wherein n has a value of 3 to 6, denotes a branched or unbranched hydrocarbon group with 2 to n C atoms and a C≡C triple bond. Examples of such groups include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl etc.

Unless otherwise stated alkynyl groups are connected to the remainder of the molecule via the C atom in position 1. Therefore terms such as 1-propynyl, 2-propynyl, 1-butynyl, etc. are equivalent to the terms 1-propyn-1-yl, 2-propyn-1-yl, 1-butyn-1-yl, etc.. This also applies analogously to C₂₋ₙ-alkenyl groups.

The term C₁₋ₙ-alkoxy denotes a C₁₋ₙ-alkyl-O group, wherein C₁₋ₙ-alkyl is as hereinbefore defined. Examples of such groups include methoxy, ethoxy, n-propoxy, iso-propoxy, n-butoxy, iso-butoxy, sec-butoxy, tert-butoxy, n-pentoxy, iso-pentoxy, neo-pentoxy, tert-pentoxy, n-hexoxy, iso-hexoxy etc.

The term C₁₋ₙ-alkylcarbonyl denotes a C₁₋ₙ-alkyl-C(=O) group, wherein C₁₋ₙ-alkyl is as hereinbefore defined. Examples of such groups include methylcarbonyl, ethylcarbonyl, n-propylcarbonyl, iso-propylcarbonyl, n-butylcarbonyl, iso-butylcarbonyl, sec-butylcarbonyl, tert-butylcarbonyl, n-pentylcarbonyl, iso-pentylcarbonyl, neo-pentylcarbonyl, tert-pentylcarbonyl, n-hexylcarbonyl, iso-hexylcarbonyl, etc.

The term C₃₋ₙ-cycloalkyl denotes a saturated mono-, bi-, tri- or spirocarbocyclic group with 3 to n C atoms. Examples of such groups include cyclopropyl, cyclobutyl, cyclopentyl, cyclohexyl, cycloheptyl, cyclooctyl, cyclononyl, cyclododecyl, bicyclo[3.2.1.]octyl, spiro[4.5]decyl, norpinyl, norbonyl, norcaryl, decalinyl, adamantyl, etc. Preferably the term C₃₋₇-cycloalkyl denotes saturated monocyclic groups.

The term C₅₋ₙ-cycloalkenyl denotes a C₅₋ₙ-cycloalkyl group which is as hereinbefore defined and additionally has at least one unsaturated C=C double bond.

The term C₃₋ₙ-cycloalkylcarbonyl denotes a C₃₋ₙ-cycloalkyl-C(=O) group wherein C₃₋ₙ-cycloalkyl is as hereinbefore defined.

The term tri-(C₁₋₄-alkyl)silyl comprises silyl groups which have identical or two or three different alkyl groups.

The term di-(C₁₋₃-alkyl)amino comprises amino groups which have identical or two different alkyl groups.

The term aryl preferably denotes naphthyl or phenyl, more preferably phenyl.

The nomenclature in structural formulas used above and hereinafter, in which a bond of a substituent of a cyclic group, as e.g. a phenyl ring, is shown towards the centre of the cyclic group, denotes, unless otherwise stated, that this substituent may be bound to any free position of the cyclic group bearing an H atom.

The compounds according to the invention may be obtained using methods of synthesis known in principle. Preferably the compounds are obtained by the following methods according to the invention which are described in more detail hereinafter.

The glucose derivatives of formula II according to the invention may be synthesised from D-gluconolactone or a derivative thereof by adding the desired benzylbenzene compound in the form of an organometallic compound (Scheme 1).

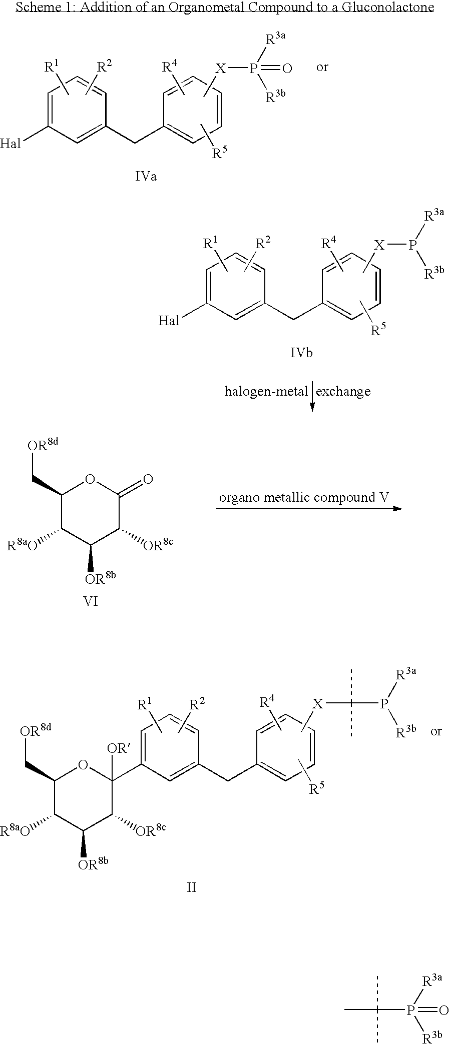

The reaction according to Scheme 1 is preferably carried out starting from a halogenated benzylbenzene compound of general formula IVa or IVb, wherein Hal denotes chlorine, bromine, or iodine. Starting from the haloaromatic compound IVa or IVb the corresponding organometallic compound (V) may be prepared either by means of a so-called halogen-metal exchange reaction or by inserting the metal into the carbon-halogen bond. The halogen-metal exchange with bromine or iodine-substituted aromatic groups may be carried out for example with an organolithium compound such as e.g. n-, sec- or tert-butyllithium and thereby yields the corresponding lithiated aromatic group. The analogous magnesium compound may also be generated by a halogen-metal exchange with a suitable Grignard reagent such as e.g. isopropylmagnesium bromide or diisopropylmagnesium. The reactions are preferably carried out between 0 and -100° C., particularly preferably between −10 and −80° C., in an inert solvent or mixtures thereof, such as for example diethyl ether, tetrahydrofuran, toluene, hexane, or methylene chloride. The magnesium or lithium compounds thus obtained may optionally be transmetalated with metal salts such as e.g. cerium trichloride, to form alternative organometal compounds (V) suitable for addition. Alternatively, the organometalic compound (V) may also be prepared by inserting a metal into the carbon-halogen bond of the haloaromatic compound IV. Metals such as e.g. lithium or magnesium are suitable for this. The addition of the organometalic compound V to gluconolactone or derivatives thereof of formula VI is preferably carried out at temperatures between 0 and −100° C., particularly preferably at −30 to −80° C., in an inert solvent or mixtures thereof, to obtain the compound of formula II. The lithiation and/or coupling reaction may also be carried out in microreactors and/or micromixers in order to avoid low temperatures; for example analogously to the processes described in WO 2004/076470. Suitable solvents for the addition of the metalated phenyl group to the appropriately protected gluconolactone are e.g. diethyl ether, toluene, methylene chloride, hexane, tetrahydrofuran or mixtures thereof. The addition reactions may be carried out without any further adjuvants or in the case of sluggishly reacting coupling partners in the presence of Lewis acids such as e.g. BF₃*OEt₂ or Me₃SiCl (see M. Schlosser, Organometallics in Synthesis, John Wiley & Sons, Chichester/New York/Brisbane/Toronto/Singapore, 1994). Preferred definitions of the groups R^{8a}, R8b, R^{8c} and R^{8d} are benzyl, substituted benzyl, trialkylsilyl, particularly preferably trimethylsilyl, triisopropylsilyl, 4-methoxybenzyl and benzyl. If two adjacent groups of the group consisting of R^{8a}, R^{8b}, R^{8c} and R^{8d} are linked together, these two groups are preferably part of a benzylideneacetal, 4-methoxybenzylideneacetal, isopropylketal or constitute a 2,3-dimethoxy-butylene group which is linked via the 2 and 3 positions of the butane with the adjacent oxygen atoms of the pyranose ring. The group R¹ preferably denotes hydrogen or C₁₋₄-alkyl, particularly preferably hydrogen, methyl or ethyl. The group R¹ is inserted after the addition of the organometallic compound V or a derivative thereof to the gluconolactone VI. For this purpose the reaction solution is treated with an alcohol such as e.g. methanol or ethanol or water in the presence of an acid such as e.g. methanesulphonic acid, toluenesulphonic acid, sulphuric acid, acetic acid, or hydrochloric acid.

The synthesis of haloaromatic compounds of formula IVa and IVb may be carried out using standard transformations in organic chemistry or at least methods known from the specialist literature in organic synthesis (see inter alia J. March, Advanced Organic Reactions, Reactions, Mechanisms, and Structure, 4th Edition, John Wiley & Sons, Chichester/New York/Brisbane/Toronto/Singapore, 1992 and literature cited therein). The residue X-P(O)R^{3a}R^{3b} or a part of it as defined herein may be introduced before, as presented above, or after the glucose moiety has been attached to the aglycon part. In principle, X-P(O)R3aR^{3b} or a part of it can be appended at any stage of the entire reaction sequence. The preferred stage of attachment, as presented above, is before the glucose part has been incorporated. In case the phosphorous atom in the residue X-P(O)R^{3a}R^{3b} is introduced with a lower oxidation state such as in X-PR^{3a}R^{3b} the phosphorous atom is oxidized at a subsequent stage of the synthesis to establish the PO moiety. Suitable oxidizing reagents are e.g. oxygen, hydrogen peroxide, tert-butyl hydrogenperoxide, 3-chloroperoxybenzoic acid, oxone, potassium monopersulfate, and benzoyl peroxide that may be used in acetone, methanol, ethanol, water, dichloromethane, acetonitrile, benzene, toluene, tetrahydrofuran, hexane, dimethylformamide, and ether at temperatures between −80° C. and 100° C., preferably between −10° C. and 60° C.

The introduction of the phosphorous containing residue can be done according to the vast number of examples reported in the organic chemistry literature. A quite general approach is the attachment via a nucleophilic substitution with an alkyl electrophile and a nucleophilic phosphorous species. Suitable alkyl electrophiles are e.g. halides such as chloride, bromide and iodide, sulfonates such as mesylate, triflate and tosylate. Alcohols may also be used as electrophiles after in situ activation of the alcoholic function; in situ activation can be achieved with e.g. strong acids or through the formation of a good leaving group as in the Mitsunobu reaction. Suitable phosphorous nucleophiles are e.g. diaryl, dialkyl or arylalkyl phosphines or their anionic counterparts that may be generated by treatment with a base. The classical Michaelis-Arbuzov reaction is a variation of this reaction principle and represents a possibility to introduce the phosphorous atom in the desired oxidation state as phosphinoxide. The reverse reactivity pattern, i.e. the phosphorous residue plays the electrophilic part and the aryl or alkyl residue of the rest of the molecule has the nucleophilic reactivity, also offers a synthetic route to the target structure. Suitable aryl or alkyl nucleophiles are e.g. the corresponding metalated compounds such as the Grignard or lithiated reagent. Phosphorous halides such as e.g. dialkylphosphanoyl chloride, alkylarylphosphanoyl chloride or diarylphosphanoyl chloride may be used as electrophiles.

In order to prepare compounds of general formula I, in process a) according to the invention, a compound of general formula II 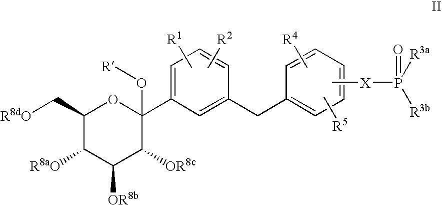
wherein R¹, X, R¹, R², R^{3a}, R^{3b}, R⁴, R⁵ are as hereinbefore defined and

R^{8a}, R8b, R^{8c}, R^{8d} are as hereinbefore defined and independently of one another represent for example acetyl, pivaloyl, benzoyl, tert-butoxycarbonyl, benzyloxycarbonyl, trialkylsilyl, benzyl or substituted benzyl or in each case two adjacent groups R^{8a}, R^{8b}b, R^{8c}, R^{8d} form a benzylideneacetal or isopropylideneketal or a 2,3-dimethoxy-butylene group which is linked via position 2 and 3 of the butylene group to the oxygen atoms of the pyranose ring and forms with them a substituted dioxane,

which may be obtained as hereinbefore described, is reacted with a reducing agent in the presence of a Lewis or Brønsted acid.

Suitable reducing agents for the reaction include for example silanes, such as triethyl-, tripropyl-, triisopropyl- or diphenylsilane, sodium borohydride, sodium cyanoborohydride, zinc borohydride, boranes, lithium aluminium hydride, diisobutylaluminium hydride or samarium iodide. The reductions are carried out without or in the presence of a suitable Brønsted acid, such as e.g. hydrochloric acid, toluenesulphonic acid, trifluoroacetic acid or acetic acid, or Lewis acid, such as e.g. boron trifluoride etherate, trimethylsilyltriflate, titaniium tetrachloride, tin tetrachloride, scandium triflate or zinc iodide. Depending on the reducing agent and the acid the reaction may be carried out in a solvent, such as for example methylene chloride, chloroform, acetonitrile, toluene, hexane, diethyl ether, tetrahydrofuran, dioxane, ethanol, water or mixtures thereof at temperatures between −60° C. and 120° C. One particularly suitable combination of reagents consists for example of triethylsilane and boron trifluoride etherate, which is conveniently used in acetonitrile or dichloromethane at temperatures of −60° C. and 60° C. Moreover, hydrogen may be used in the presence of a transition metal catalyst, such as e.g. palladium on charcoal or Raney nickel, in solvents such as tetrahydrofuran, ethyl acetate, methanol, ethanol, water or acetic acid, for the transformation described.

Alternatively, in order to prepare compounds of general formula I according to process b) according to the invention, in a compound of general formula III 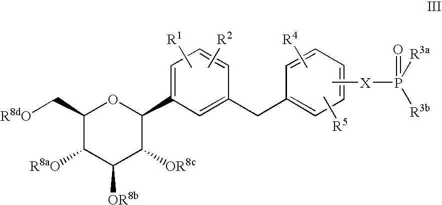
wherein R¹, R², R^{3a}, R^{3b}, R⁴, R⁵ are as hereinbefore defined and

R^{8a} to R^{8d} denote one of the protective groups defined hereinbefore, such as e.g. an acyl, arylmethyl, acetal, ketal or silyl group, and which may be obtained for example by reduction from the compound of formula II as hereinbefore described, the protective groups are cleaved.

Any acyl protecting group used is cleaved for example hydrolytically in an aqueous solvent, e.g. in water, isopropanol/water, acetic acid/water, tetrahydrofuran/water or dioxane/water, in the presence of an acid such as trifluoroacetic acid, hydrochloric acid or sulphuric acid or in the presence of an alkali metal base such as lithium hydroxide, sodium hydroxide or potassium hydroxide or aprotically, e.g. in the presence of iodotrimethylsilane, at temperatures between 0 and 120° C., preferably at temperatures between 10 and 100° C. A trifluoroacetyl group is preferably cleaved by treating with an acid such as hydrochloric acid, optionally in the presence of a solvent such as acetic acid at temperatures between 50 and 120° C. or by treating with sodium hydroxide solution optionally in the presence of a solvent such as tetrahydrofuran or methanol at temperatures between 0 and 50° C.

Any acetal or ketal protecting group used is cleaved for example hydrolytically in an aqueous solvent, e.g. in water, isopropanol/water, acetic acid/water, tetrahydrofuran/water or dioxane/water, in the presence of an acid such as trifluoroacetic acid, hydrochloric acid or sulphuric acid or aprotically, e.g. in the presence of iodotrimethylsilane, at temperatures between 0 and 120° C., preferably at temperatures between 10 and 100° C.

A trimethylsilyl group is cleaved for example in water, an aqueous solvent mixture or a lower alcohol such as methanol or ethanol in the presence of a base such as lithium hydroxide, sodium hydroxide, potassium carbonate or sodium methoxide.

In aqueous or alcoholic solvents, acids such as e.g. hydrochloric acid, trifluoroacetic acid or acetic acid are also suitable. For cleaving in organic solvents, such as for example diethyl ether, tetrahydrofuran or dichloromethane, it is also suitable to use fluoride reagents, such as e.g. tetrabutylammonium fluoride.

A benzyl, methoxybenzyl or benzyloxycarbonyl group is advantageously cleaved hydrogenolytically, e.g. with hydrogen in the presence of a catalyst such as palladium/charcoal in a suitable solvent such as methanol, ethanol, ethyl acetate or glacial acetic acid, optionally with the addition of an acid such as hydrochloric acid at temperatures between 0 and 100° C., but preferably at ambient temperatures between 20 and 60° C., and at a hydrogen pressure of 1 to 7 bar, but preferably 3 to 5 bar. A 2,4-dimethoxybenzyl group, however, is preferably cleaved in trifluoroacetic acid in the presence of anisole.

A tert.butyl or tert.butyloxycarbonyl group is preferably cleaved by treating with an acid such as trifluoroacetic acid or hydrochloric acid or by treating with iodotrimethylsilane optionally using a solvent such as methylene chloride, dioxane, methanol or diethylether.

In the reactions described hereinbefore, any reactive groups present such as ethynyl, hydroxy, amino, alkylamino or imino groups may be protected during the reaction by conventional protecting groups which are cleaved again after the reaction.

For example, a protecting group for an ethynyl group may be the trimethylsilyl or triisopropyl group. The 2-hydroxisoprop-2-yl group may also be used as a protective group.

For example, a protecting group for a hydroxy group may be a trimethylsilyl, acetyl, trityl, benzyl or tetrahydropyranyl group.

Protecting groups for an amino, alkylamino or imino group may be, for example, a formyl, acetyl, trifluoroacetyl, ethoxycarbonyl, tert.butoxycarbonyl, benzyloxycarbonyl, benzyl, methoxybenzyl or 2,4-dimethoxybenzyl group.

Moreover, the compounds of general formula I obtained may be resolved into their enantiomers and/or diastereomers, as mentioned hereinbefore. Thus, for example, cis/trans mixtures may be resolved into their cis and trans isomers, and compounds with at least one optically active carbon atom may be separated into their enantiomers.

Thus, for example, the cis/trans mixtures may be resolved by chromatography into the cis and trans isomers thereof, the compounds of general formula I obtained which occur as racemates may be separated by methods known per se (cf. Allinger N. L. and Eliel E. L. in “Topics in Stereochemistry”, Vol. 6, Wiley Interscience, 1971) into their optical antipodes and compounds of general formula I with at least 2 asymmetric carbon atoms may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

The enantiomers are preferably separated by column chromatography on chiral phases or by recrystallisation from an optically active solvent or by reacting with an optically active substance which forms salts or derivatives such as e.g. esters or amides with the racemic compound, particularly acids and the activated derivatives or alcohols thereof, and separating the diastereomeric mixture of salts or derivatives thus obtained, e.g. on the basis of their differences in solubility, whilst the free antipodes may be released from the pure diastereomeric salts or derivatives by the action of suitable agents. Optically active acids in common use are e.g. the D- and L-forms of tartaric acid or dibenzoyltartaric acid, di-o-tolyltartaric acid, malic acid, mandelic acid, camphorsulfonic acid, glutamic acid, aspartic acid or quinic acid. An optically active alcohol may be for example (+) or (−)-menthol and an optically active acyl group in amides, for example, may be a (+)-or (−)-menthyloxycarbonyl.

Furthermore, the compounds of formula I may be converted into the salts thereof, particularly for pharmaceutical use into the physiologically acceptable salts with inorganic or organic acids. Acids which may be used for this purpose include for example hydrochloric acid, hydrobromic acid, sulphuric acid, methanesulphonic acid, phosphoric acid, fumaric acid, succinic acid, lactic acid, citric acid, tartaric acid or maleic acid.

Moreover, the compounds obtained may be converted into mixtures, for example 1:1 or 1:2 mixtures with amino acids, particularly with alpha-amino acids such as proline or phenylalanine, which may have particularly favourable properties such as a high crystallinity.

The compounds according to the invention are advantageously also obtainable using the methods described in the examples that follow, which may also be combined for this purpose with methods known to the skilled man from the literature, for example, particularly the methods described in WO 98/31697, WO 01/27128, WO 02/083066, WO 03/099836 and WO 2004/063209.

As already mentioned, the compounds of general formula I according to the invention and the physiologically acceptable salts thereof have valuable pharmacological properties, particularly an inhibitory effect on the sodium-dependent glucose cotransporter SGLT, preferably SGLT2.

The biological properties of the new compounds may be investigated as follows:

The ability of the substances to inhibit the SGLT-2 activity may be demonstrated in a test set-up in which a CHO-K1 cell line (ATCC No. CCL 61) or alternatively an HEK293 cell line (ATCC No. CRL-1573), which is stably transfected with an expression vector pZeoSV (Invitrogen, EMBL accession number L36849) , which contains the cDNA for the coding sequence of the human sodium glucose cotransporter 2 (Genbank Acc. No.NM_{—}003041) (CHO-hSGLT2 or HEK-hSGLT2). These cell lines transport ¹⁴C-labelled alpha-methyl-glucopyranoside (¹⁴C-AMG, Amersham) into the interior of the cell in sodium-dependent manner.

The SGLT-2 assay is carried out as follows:

CHO-hSGLT2 cells are cultivated in Ham's F12 Medium (BioWhittaker) with 10% foetal calf serum and 250 μg/mL zeocin (Invitrogen), and HEK293-hSGLT2 cells are cultivated in DMEM medium with 10% foetal calf serum and 250 μg/mL zeocin (Invitrogen). The cells are detached from the culture flasks by washing twice with PBS and subsequently treating with trypsin/EDTA. After the addition of cell culture medium the cells are centrifuged, resuspended in culture medium and counted in a Casy cell counter. Then 40,000 cells per well are seeded into a white, 96-well plate coated with poly-D-lysine and incubated overnight at 37° C., 5% CO₂. The cells are washed twice with 250 μl of assay buffer (Hanks Balanced Salt Solution, 137 mM NaCl, 5.4 mM KCl, 2.8 mM CaCl₂, 1.2 mM MgSO₄ and 10 mM HEPES (pH 7.4), 50 μg/mL of gentamycin). 250 μl of assay buffer and 5 μl of test compound are then added to each well and the plate is incubated for a further 15 minutes in the incubator. 5 μl of 10% DMSO are used as the negative control. The reaction is started by adding 5 μl of ¹⁴C-AMG (0.05 μCi) to each well. After 2 hours' incubation at 37° C., 5% CO₂, the cells are washed again with 250 μl of PBS (20° C.) and then lysed by the addition of 25 μl of 0.1 N NaOH (5 min. at 37° C.). 200 μl of MicroScint20 (Packard) are added to each well and incubation is continued for a further 20 min at 37° C. After this incubation the radioactivity of the ¹⁴C-AMG absorbed is measured in a Topcount (Packard) using a ¹⁴C scintillation program.

To determine the selectivity with respect to human SGLT1 an analogous test is set up in which the cDNA for hSGLT1 (Genbank Acc. No. NM000343) instead of hSGLT2 cDNA is expressed in CHO-K1 or HEK293 cells.

The compounds of general formula I according to the invention may for example have EC50 values below 1000 nM, particularly below 200 nM, most preferably below 50 nM.

In view of their ability to inhibit the SGLT activity, the compounds of general formula I according to the invention and the corresponding pharmaceutically acceptable salts thereof are theoretically suitable for the treatment and/or preventative treatment of all those conditions or diseases which may be affected by the inhibition of the SGLT activity, particularly the SGLT-2 activity. Therefore, compounds according to the invention are particularly suitable for the prevention or treatment of diseases, particularly metabolic disorders, or conditions such as type 1 and type 2 diabetes mellitus, complications of diabetes (such as e.g. retinopathy, nephropathy or neuropathies, diabetic foot, ulcers, macroangiopathies), metabolic acidosis or ketosis, reactive hypoglycaemia, hyperinsulinaemia, glucose metabolic disorder, insulin resistance, metabolic syndrome, dyslipidaemias of different origins, atherosclerosis and related diseases, obesity, high blood pressure, chronic heart failure, edema and hyperuricaemia. These substances are also suitable for preventing beta-cell degeneration such as e.g. apoptosis or necrosis of pancreatic beta cells. The substances are also suitable for improving or restoring the functionality of pancreatic cells, and also of increasing the number and size of pancreatic beta cells. The compounds according to the invention may also be used as diuretics or antihypertensives and are suitable for the prevention and treatment of acute renal failure.

In particular, the compounds according to the invention, including the physiologically acceptable salts thereof, are suitable for the prevention or treatment of diabetes, particularly type 1 and type 2 diabetes mellitus, and/or diabetic complications.

The dosage required to achieve the corresponding activity for treatment or prevention usually depends on the compound which is to be administered, the patient, the nature and gravity of the illness or condition and the method and frequency of administration and is for the patient's doctor to decide. Expediently, the dosage may be from 1 to 100 mg, preferably 1 to 30 mg, by intravenous route, and 1 to 1000 mg, preferably 1 to 100 mg, by oral route, in each case administered 1 to 4 times a day. For this purpose, the compounds of formula I prepared according to the invention may be formulated, optionally together with other active substances, together with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, powders, suspensions or suppositories.

The compounds according to the invention may also be used in conjunction with other active substances, particularly for the treatment and/or prevention of the diseases and conditions mentioned above. Other active substances which are suitable for such combinations include for example those which potentiate the therapeutic effect of an SGLT antagonist according to the invention with respect to one of the indications mentioned and/or which allow the dosage of an SGLT antagonist according to the invention to be reduced. Therapeutic agents which are suitable for such a combination include, for example, antidiabetic agents such as mefformin, sulphonylureas (e.g. glibenclamide, tolbutamide, glimepiride), nateglinide, repaglinide, thiazolidinediones (e.g. rosiglitazone, pioglitazone), PPAR-gamma-agonists (e.g. GI 262570) and antagonists, PPAR-gamma/alpha modulators (e.g. KRP 297), alpha-glucosidase inhibitors (e.g. acarbose, voglibose), DPPIV inhibitors (e.g. LAF237, MK431), alpha2-antagonists, insulin and insulin analogues, GLP-1 and GLP-1 analogues (e.g. exendin4) or amylin. The list also includes inhibitors of protein tyrosinephosphatase 1, substances that affect deregulated glucose production in the liver, such as e.g. inhibitors of glucose-6-phosphatase, or fructose-1,6-bisphosphatase, glycogen phosphorylase, glucagon receptor antagonists and inhibitors of phosphoenol pyruvate carboxykinase, glycogen synthase kinase or pyruvate dehydrokinase, lipid lowering agents such as for example HMG-CoA-reductase inhibitors (e.g. simvastatin, atorvastatin), fibrates (e.g. bezafibrate, fenofibrate), nicotinic acid and the derivatives thereof, PPAR-alpha agonists, PPAR-delta agonists, ACAT inhibitors (e.g. avasimibe) or cholesterol absorption inhibitors such as, for example, ezetimibe, bile acid-binding substances such as, for example, cholestyramine, inhibitors of ileac bile acid transport, HDL-raising compounds such as CETP inhibitors or ABC1 regulators or active substances for treating obesity, such as sibutramine or tetrahydrolipostatin, dexfenfluramine, axokine, antagonists of the cannabinoid1 receptor, MCH-1 receptor antagonists, MC4 receptor agonists, NPY5 or NPY2 antagonists or β3-agonists such as SB418790 or AD-9677 and agonists of the 5HT2c receptor.

Moreover, combinations with drugs for influencing high blood pressure, chronic heart failure or atherosclerosis such as e.g. A-II antagonists or ACE inhibitors, ECE inhibitors, diuretics, β-blockers, Ca-antagonists, centrally acting antihypertensives, antagonists of the alpha-2-adrenergic receptor, inhibitors of neutral endopeptidase, thrombocyte aggregation inhibitors and others or combinations thereof are suitable. Examples of angiotensin 11 receptor antagonists are candesartan cilexetil, potassium losartan, eprosartan mesylate, valsartan, telmisartan, irbesartan, EXP-3174, L-158809, EXP-3312, olmesartan, medoxomil, tasosartan, KT-3-671, GA-0113, RU-64276, EMD-90423, BR-9701, etc. Angiotensin II receptor antagonists are preferably used for the treatment or prevention of high blood pressure and complications of diabetes, often combined with a diuretic such as hydrochlorothiazide.

A combination with uric acid synthesis inhibitors or uricosurics is suitable for the treatment or prevention of gout.

A combination with GABA-receptor antagonists, Na-channel blockers, topiramat, protein-kinase C inhibitors, advanced glycation end product inhibitors or aldose reductase inhibitors may be used for the treatment or prevention of complications of diabetes.

The dosage for the combination partners mentioned above is usefully ⅕ of the lowest dose normally recommended up to 1/1 of the normally recommended dose.

Therefore, in another aspect, this invention relates to the use of a compound according to the invention or a physiologically acceptable salt of such a compound combined with at least one of the active substances described above as a combination partner, for preparing a pharmaceutical composition which is suitable for the treatment or prevention of diseases or conditions which can be affected by inhibiting the sodium-dependent glucose cotransporter SGLT. These are preferably metabolic diseases, particularly one of the diseases or conditions listed above, most particularly diabetes or diabetic complications.

The use of the compound according to the invention, or a physiologically acceptable salt thereof, in combination with another active substance may take place simultaneously or at staggered times, but particularly within a short space of time. If they are administered simultaneously, the two active substances are given to the patient together; while if they are used at staggered times the two active substances are given to the patient preferably within a period of less than or equal to 12 hours, but particularly less than or equal to 6 hours.

Consequently, in another aspect, this invention relates to a pharmaceutical composition which comprises a compound according to the invention or a physiologically acceptable salt of such a compound and at least one of the active substances described above as combination partners, optionally together with one or more inert carriers and/or diluents.

Thus, for example, a pharmaceutical composition according to the invention comprises a combination of a compound of formula I according to the invention or a physiologically acceptable salt of such a compound and at least one angiotensin II receptor antagonist optionally together with one or more inert carriers and/or diluents.

The compound according to the invention, or a physiologically acceptable salt thereof, and the additional active substance to be combined therewith may both be present together in one formulation, for example a tablet or capsule, or separately in two identical or different formulations, for example as a so-called kit-of-parts.

In the foregoing and following text, H atoms of hydroxyl groups are not explicitly shown in every case in structural formulae. The Examples that follow are intended to illustrate the present invention without restricting it:

Preparation of the starting compounds:

### EXAMPLE I

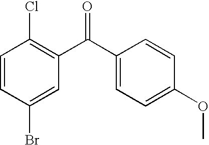

### (5-Bromo-2-chloro-phenyl)-(4-methoxy-phenyl)-methanone

38.3 mL oxalyl chloride and 0.8 mL dimethylformamide are added to a mixture of 100 g 5-bromo-2-chloro-benzoic acid in 500 mL dichloromethane. The reaction mixture is stirred for 14 h, then filtered, and separated from all volatile constituents in a rotary evaporator. The residue is dissolved in 150 mL dichloromethane, the solution is cooled to −5° C., and 46.5 g anisole are added. Then 51.5 g aluminum trichloride are added batchwise so that the temperature does not exceed 5° C. The solution is stirred for 1 h at 1-5° C. and then poured onto crushed ice. The organic phase is separated off, and the aqueous phase is extracted with dichloromethane. The combined organic phases are washed with aqueous 1 M hydrochloric acid, twice with 1 M sodium hydroxide solution, and with brine. Then the organic phase is dried over sodium sulfate, the solvent is removed, and the residue is recrystallized from ethanol.

Yield: 86.3 9 (64% of theory) Mass spectrum (ESI⁺): m/z=325/327/329 (Br+Cl) [M+H]⁺

The following compound may be obtained analogously to Example I:

(1) (5-Bromo-2-methyl-phenyl)-(4-methoxy-phenyl)-methanone

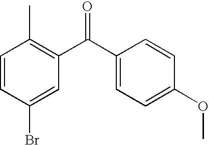

Mass spectrum (ESI⁺): m/z=305/307 (Br) [M+H]⁺

### EXAMPLE II

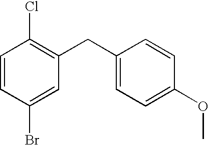

### 4-Bromo-1 -chloro-2-(4-methoxv-benzyl)-benzene

A solution of 86.2 g (5-bromo-2-chloro-phenyl)-(4-methoxy-phenyl)-methanone and 101.5 mL triethylsilane in 75 mL dichloromethane and 150 mL acetonitrile is cooled to 10° C. Then with stirring 50.8 mL of boron trifluoride etherate are added so that the temperature does not exceed 20° C. The solution is stirred for 14 h at ambient temperature, before another 9 mL triethylsilane and 4.4 mL boron trifluoride etherate are added. The solution is stirred for a further 3 h period at 45-50° C. and then cooled to ambient temperature. A solution of 28 g potassium hydroxide in 70 mL water is added, and the resultant mixture is stirred for 2 h. The organic phase is separated off, and the aqueous phase is extracted another three times with diisopropylether. The combined organic phases are washed twice with 2 M potassium hydroxide solution and once with brine and then dried over sodium sulfate. After the solvent is evaporated, the residue is stirred in ethanol, separated again, and dried at 60° C.

Yield: 50.0 g (61% of theory) Mass spectrum (ESI⁺): m/z=310/312/314 (Br+Cl) [M+H]⁺

The following compound may be obtained analogously to Example II

(1) 4-bromo-1-methyl-2-(4-methoxy-benzyl)-benzene

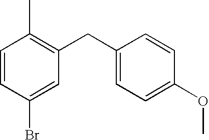

Mass spectrum (EI): m/z=290/292 (Br) [M]⁺

### EXAMPLE III

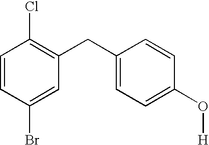

### 4-(5-Bromo-2-chloro-benzyl)-phenol

A solution of 14.8 g 4-bromo-1-chloro-2-(4-methoxy-benzyl)-benzene in 150 mL dichloromethane is cooled in an ice bath. 50 mL of a 1 M solution of boron tribromide in dichloromethane are added, and the resulting solution is stirred for 2 h at ambient temperature. The solution is then cooled in an ice bath again, and saturated aqueous potassium carbonate solution is added dropwise. At ambient temperature the mixture is adjusted with aqueous 1 M hydrochloric acid to pH 1, the organic phase is separated off, and the aqueous phase is extracted three times with ethyl acetate. The combined organic phases are dried over sodium sulfate, and the solvent is removed completely.

Yield: 13.9 g (98% of theory) Mass spectrum (ESI⁻): m/z=295/297/299 (Br+Cl) [M-H]⁻

The following compound may be obtained analogously to Example III

(1) 4-(5-Bromo-2-methyl-benzyl)-phenol

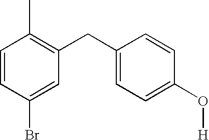

Mass spectrum (ESI⁻): m/z=275/277 (Br) [M-H]⁻

### EXAMPLE IV

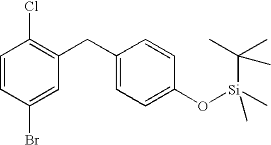

### [4-(5-Bromo-2-chloro-benzyl)-Phenoxyl-tert-butyl-dimethyl-silane

A solution of 13.9 g 4-(5-bromo-2-chloro-benzyl)-phenol in 140 mL dichloromethane is cooled in an ice bath. Then 7.54 g tert-butyldimethylsilyl chloride in 20 mL dichloromethane are added followed by 9.8 mL triethylamine and 0.5 g dimethylaminopyridine. The resultant solution is stirred for 16 h at ambient temperature and then diluted with 100 mL dichloromethane. The organic phase is washed twice with aqueous 1 M hydrochloric acid and once with aqueous sodium hydrogen carbonate solution and then dried over sodium sulfate. After the solvent is removed, the residue is filtered through silica gel (cyclohexane/ethyl acetate 100:1).

Yield: 16.8 g (87% of theory) Mass spectrum (EI): m/z=410/412/414 (Br+Cl) [M]⁺

The following compound may be obtained analogously to Example IV

(1) [4-(5-Bromo-2-methyl-benzyl)-phenoxy]-tert-butyl-dimethyl-silane

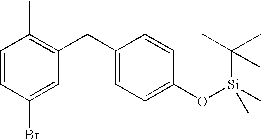

Mass spectrum (ESI⁺): m/z=391/393 (Br) [M+H]⁺

### EXAMPLE V

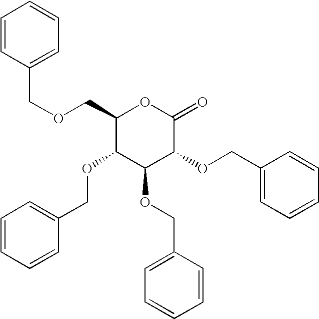

### 2,3,4,6-Tetra-O-benzyl-D-glucopyranone

To a solution of 10.0 g 2,3,4,6-tetra-O-benzyl-α-D-glucopyranose in 140 mL dichloromethane are added 4 g freshly activated molecular sieves 4 Å and 3.3 g N-methylmorpholine-N-oxide. The solution is stirred for 20 min at ambient temperature, before 0.3 g tetra-n-propylammonium perruthenate are added. After 2 h stirring at ambient temperature the solution is diluted with dichloromethane and filtered through Celite. The filtrate is washed with aqueous sodium thiosulfate solution and water and then dried over sodium sulfate. After the solvent is evaporated, the residue is chromatographed on silica gel (cyclohexane/ethyl acetate 4:1).

Yield: 8.2 g (82% of theory) Mass spectrum (ESI⁺): m/z=539 [M+H]⁺

### EXAMPLE VI

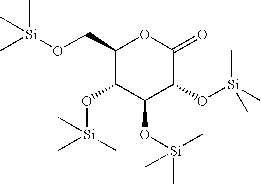

### 2,3,4,6-Tetrakis-O-(trimethylsilyl)-D-glucopyranone

A solution of 20 g D-glucono-1,5-lactone and 98.5 mL N-methylmorpholine in 200 mL of tetrahydrofuran is cooled to −5° C. Then 85 mL trimethylsilyl chloride are added dropwise so that the temperature does not exceed 5° C. The solution is then stirred for 1 h at ambient temperature, 5 h at 35° C. and again for 14 h at ambient temperature. After the addition of 300 mL of toluene the solution is cooled in an ice bath, and 500 mL of water are added so that the temperature does not exceed 10° C. The organic phase is then separated and washed with aqueous sodium dihydrogen phosphate solution, water, and brine. The solvent is removed, the residue is taken up in 250 mL toluene, and the solvent is again removed completely.

Yield: 52.5 g (approx. 90% pure) Mass spectrum (ESI⁺): m/z=467 [M+H]⁺

### EXAMPLE VII

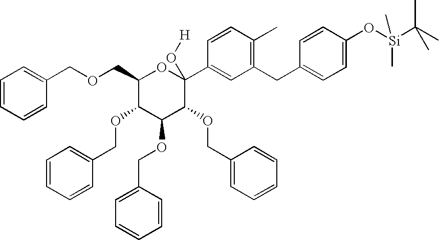

### 1-(2,3,4,6-Tetra-O-benzyl-1 -hydroxy-D-glucopyranos-1 -yl)-3-[4-(tert-butyl-dimethyl-silvioxy)-benzyl]4-methyl-benzene

A solution of 0.34 g [4-(5-bromo-2-methyl-benzyl)-phenoxy]-tert-butyl-dimethyl-silane in 3 mL dry tetrahydrofuran is cooled to −80° C. under argon. 0.54 mL of a 1.6 M solution of n-butyllithium in hexane are added dropwise, and the resulting solution is stirred for 1.5 h at −78° C. A solution of 0.43 g 2,3,4,6-tetra-O-benzyl-D-glucopyranone in 2.5 mL of tetrahydrofuran chilled to −80° C. is added dropwise to this solution by means of a transfer needle. The resulting solution is stirred for 5 h at −78° C. The reaction is quenched with a solution of 0.1 mL acetic acid in 1 mL of tetrahydrofuran and warmed to ambient temperature. Then aqueous sodium hydrogen carbonate solution is added, and the mixture is extracted four times with ethyl acetate. The organic phases are dried over sodium sulfate, and the solvent is evaporated. The residue is purified by chromatography on silica gel (cyclohexane/ethyl acetate 15:1->4:1).

Yield: 0.48 g (approx. 88% pure) Mass spectrum (ESI⁺): m/z=868 [M+H]⁺

### EXAMPLE VIII

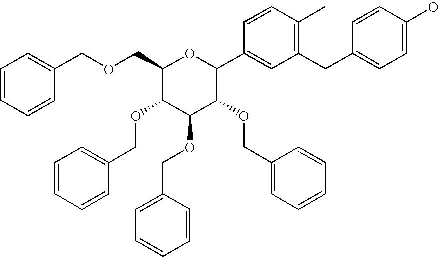

### 1-(2, 3,4,6-tetra-O-benzyl-βD-glucopyranos-1-yl)-3-(4-hydroxy-benzyl)4-methyl-benzene

A solution of 0.48 g (approx. 88% pure) 1-(2,3,4,6-tetra-O-benzyl-1-hydroxy-D-glucopyranosyl)-3-[4-(tert-butyl-dimethyl-silyloxy)-benzyl]4-methyl-benzene in 3.5 mL dry acetonitrile is cooled to −40° C. under argon. 0.13 mL triisopropylsilane and 0.08 mL boron trifluoride etherate are added dropwise. The solution is stirred for 3 h at −35° C., before another 0.02 mL of triisopropylsilane and 0.01 mL of boron trifluoride etherate are added. After a further 2 h at −40° C. aqueous potassium carbonate solution is added, and the resulting mixture is stirred for 1 h at ambient temperature. Then water is added, and the mixture is extracted four times with ethyl acetate. The organic phase is dried over sodium sulfate, concentrated, and chromatographed on silica gel (cyclohexane/ethyl acetate 10:1->4:1).

Yield: 0.24 g (68% of theory). Mass spectrum (ESI⁺): m/z=738 (M+NH₄]⁺

### EXAMPLE IX

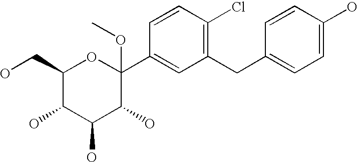

### 1 -Chloro4-(1 -methoxy-D-glucopyranos-1 -yl)-2-(4-hydroxy-benzyl)-benzene

A solution of 14.0 g [4-(5-bromo-2-chloro-benzyl)-phenoxy]-tert-butyl-dimethyl-silane in 150 mL hexane and 30 mL tetrahydrofuran is cooled to −80° C. under argon atmosphere. 11.8 mL of a −70° C.-cold solution of tert-butyllithium in pentane (1.7 M) are added dropwise to the bromobenzene solution, and the resulting solution is stirred for 45 min at −80° C. Then a −70° C.-cold solution of 18.1 g of 2,3,4,6-tetrakis-O-(trimethylsilyl)-D-glucopyranone in 50 mL hexane is added. The resulting solution is stirred for 1 h at −70° C. 150 mL 1% aqueous acetic acid solution is added, and the cooling bath is removed. After the reaction solution is warmed to room temperature, the organic phase is separated, and the aqueous phase is extracted with ethyl acetate. After drying the combined organic phases over sodium sulfate, the solvent is evaporated, and the residue is dissolved in 150 mL methanol. The resultant solution is treated with 1 mL methanesulfonic acid and stirred at room temperature for 16 h. The reaction solution is neutralized with aqueous sodium bicarbonate solution, most of the methanol is evaporated, and the aqueous residue is extracted with ethyl acetate. The combined organic phases are dried over sodium sulfate, and the solvent is evaporated. The residue is dissolved in as little methanol and ethyl acetate as possible, and the resulting solution is added to petrol ether. The precipitate is separated by filtration and dried at 50° C.

Yield: 10.0 g (72% of theory) Mass spectrum (ESI⁺): m/z=433/435 (Cl) [M+Na]⁺

The following compound may be obtained analogously to Example IX:

(1)1 -Methyl4-(1 -methoxy-D-glucopyranos-1 -yl)-2-(4-hydroxy-benzyl)-benzene

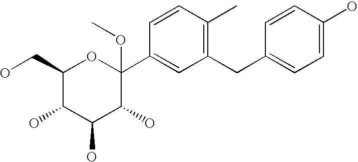

Mass spectrum (ESI⁻): m/z=389 [M-H]⁻

### EXAMPLE X

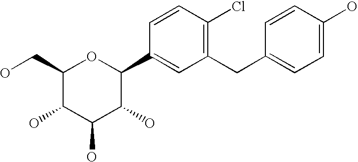

### 1 -Chloro4-(β-D-glucopyranos-1-yl)-2-(4-hydroxy-benzyl)-benzene

A solution of 25.0 g 1-chloro4-(1-methoxy-D-glucopyranos-1-yl)-2-(4-hydroxy-benzyl)-benzene and 20.0 mL triethylsilane in 120 mL dichloromethane and 360 mL acetonitrile is cooled to −5-−10° C. 10.0 mL Boron trifluoride etherate are added dropwise, and the solution is stirred in the cooling bath for 1 h. Aqueous sodium hydrogen carbonate solution is added, the organic phase is separated, and the aqueous phase is extracted with ethyl acetate. The combined organic phases are dried over sodium sulfate, and the solvent is removed in vacuo. The residue is washed with diisopropylether and dissolved in as little ethyl acetate as needed. The resulting solution is treated with cyclohexane, and the precipitate is separated by filtration and dried at 50° C.

Yield: 23.0 g (99% of theory, ca. 7:1 mixture with α-anomer) Mass spectrum (ESI⁻): m/z=425/427 (Cl) [M+HCOO]⁻

The following compound may be obtained analogously to Example X:

(1) 1-Methyl-4-(β3-D-glucopyranos-1-yl)-2-(4-hydroxy-benzyl)-benzene

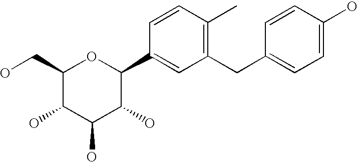

Mass spectrum (ESI⁺): m/z=378 [M+NH₄]⁺K +

### EXAMPLE XI

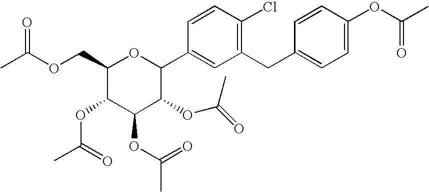

### 1 -Chloro-4-(2,3.4,6-tetra-O-acetyl-β-D-glucopyranos-1-yl)-2-(4-acetoxy-benzyl)-benzene

To a solution of 23.0 g 1-chloro4-(β-D-glucopyranos-1-yl)-2-(4-hydroxy-benzyl)-benzene and 55 mL pyridine in 200 mL dichloromethane is added 60 mL acetic acid anhydride followed by 0.1 g 4-dimethylaminopyridine. The solution is stirred for 1 h at ambient temperature. Then, the solution is diluted with dichloromethane and washed with 2 M aqueous hydrochloric acid. The organic phase is dried over sodium sulfate, and the solvent is evaporated. The residue is recrystallized from ethanol to give the pure β-anomer as a white solid.

Yield: 7.8 g (22% of theory) Mass spectrum (ESI⁺): m/z=608/610 (Cl) [M+NH₄]⁺

The following compound may be obtained analogously to Example XI:

(1)1 -Methyl4-(2,3,4,6-tetra-O-acetyl-β-D-glucopyranos-1-yl)-2-(4-acetoxy-benzyl)-benzene

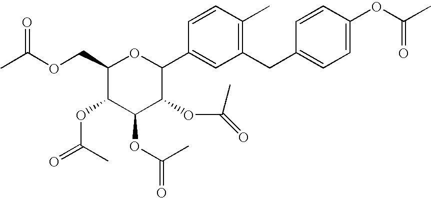

Mass spectrum (ESI⁺): m/z=588 [M+NH₄]⁺

### EXAMPLE XII

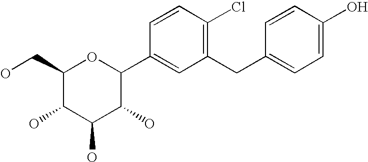

### 1 -Chloro4-(βD-glucopyranos-1-yl)-2-(4-hydroxybenzyl)-benzene

To a solution of 7.9 g 1-cloro4-(2,3,4,6-tetra-O-acetyl-β-D-glucopyranos-1-yl)-2-(4-acetoxy-benzyl)-benzene in 150 mL methanol is added 25 mL 4 M aqueous potassium hydroxide solution. The solution is stirred at room temperature for 1 h and then adjusted to pH 5 with 4 M hydrochloric acid. Most of the methanol is evaporated, and the remaining solution is extracted with ethyl acetate. The combined extracts are dried over sodium sulfate, and the solvent is removed in vacuo.

Yield: 5.1 g (100% of theory) Mass spectrum (ESI⁺): m/z=398/400 (Cl) [M+NH₄]⁺

The following compound may be obtained analogously to Example XII:

(1) 1-Methyl4-(β-D-glucopyranos-1-yl)-2-(4-hydroxy-benzyl)-benzene

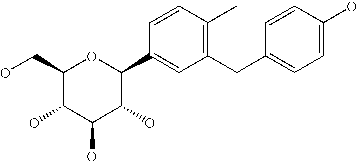

Mass spectrum (ESI⁺): m/z=378 (M+NH₄]⁺

### EXAMPLE XIII

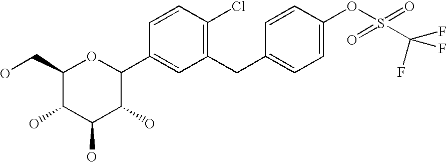

### 1 -Chloro-4-(β-D-glucopyranos-1-yl)-2-[4-(trifluoromethylsulfonyloxy)-benzyl]-benzene

10 mg 4-dimethylaminopyridine are added to a solution of 0.38 g 1-chloro-4-(β-D-glucopyranos-1-yl)-2-(4-hydroxybenzyl)-benzene, 0.21 ml triethylamine and 0.39 g N,N-bis-(trifluoromethanesulfonyl)-aniline in 10 mL dry dichloromethane. The solution is stirred for 4 h at ambient temperature and then combined with aqueous sodium chloride solution. It is extracted with ethyl acetate, the organic extracts are dried over sodium sulfate, and the solvent is removed. The residue is chromatographed through silica gel (dichloromethane/methanol 1:0->4:1).

Yield: 0.33 g (64% of theory) Mass spectrum (ESI⁺): m/z=530/532 (Cl) [M+NH₄]⁺

### EXAMPLE XIV

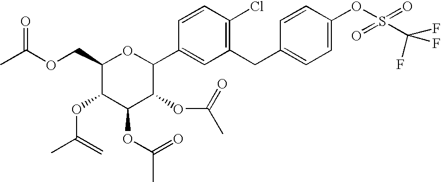

### 1-Chloro-4-(2,3,4,6-tetra-O-acetyl-β-D-glucopyranos-1-yl)-2-[4-(trifluoromethylsulfonyloxy)-benzyl]-benzene

To a solution of 3.0 g 1-chloro4-(β-D-glucopyranos-1-yl)-2-[4-(trifluoromethylsulfonyloxy)-benzyl]-benzene in 50 mL dichloromethane are added 3.8 mL pyridine, 4.1 mL acetic anhydride, and 60 mg 4-dimethylaminopyridine. The solution is stirred for 1 h at ambient temperature. 50 mL water is added, and the resulting solution stirred for an additional 5 min. The aqueous layer is separated, and the organic phase is washed with 1 M hydrochloric acid and aqueous sodium hydrogencarbonate solution and dried over magnesium sulfate. After removal of the solvent in vacuo the product is obtained as a white solid.

Yield: 3.0 g (75% of theory) Mass spectrum (ESI⁺): m/z=698/700 (Cl) [M+NH₄]⁺

### EXAMPLE XV

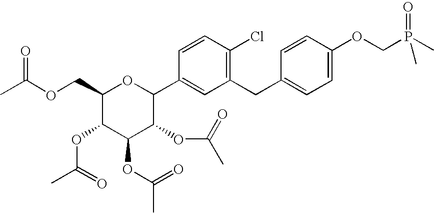

### 1 -Chloro4-(2,3,4,6-tetra-O-acetyl-β-D-glucopyranos-1-yl)-2-[4-(dimethylphosphinoylmethoxy)-benzyl]-benzene

To a stirred mixture of 1.0 g 1-chloro4-(β-D-glucopyranos-1-yl)-2-(4-hydroxy-benzyl)-benzene and 1.0 g potassium carbonate in 10 mL dimethylformamide is added 0.6 g chloro-(dimethylphosphanoyl)-methane. The mixture is stirred over night at 80° C. After cooling to ambient temperature, the reaction mixture is neutralized with 1 M hydrochloric acid, and the solvent is evaporated. The residue is taken up in 10 mL dichloromethane, and to the resultant suspension are added 2.1 mL pyridine, 2.4 mL acetic anhydride, and 50 mg 4-dimethylaminopyridine. The solution is stirred for 1 h at ambient temperature. 50 mL water is added, and the resulting solution stirred for an additional 5 min. The aqueous layer is separated, and the organic phase is washed with 1 M hydrochloric acid and aqueous sodium hydrogencarbonate solution and dried over magnesium sulfate. After removal of the solvent in vacuo, the residue is purified by chromatography on silica gel (dichloromethane/methanol 9: 1->2:1) to furnish the product as a white solid.

Yield: 0.65 g (39% of theory) Mass spectrum (ESI⁺): m/z=639/641 (Cl) [M+H]⁺

### EXAMPLE XVI

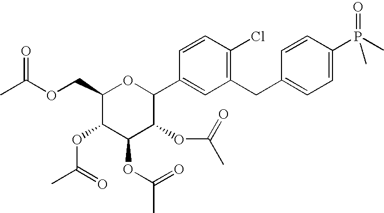

### 1 -Chloro4-(2, 3,4,6-tetra-O-acetyl-β-D-glucopyranos-1-yl)-2-[4-(dimethylphosphinoyl)-benzyl]-benzene

To a stirred mixture of 0.3 g 1-chloro4-(2,3,4,6-tetra-O-acetyl-β-D-glucopyranos-1-yl)-2-[4-(trifluoromethylsulfonyloxy)-benzyl]-benzene, 70 mg dimethylphosphinoylchloride, 10 mg palladium acetate, and 19 mg 1,4-bis(diphenylphosphino)butane in 1 mL dimethylsulfoxide and 0.5 mL toluene under argon atmosphere is added 0.15 mL ethyldiisopropylamine. The reaction mixture is stirred for 24 h at 110° C. After cooling to ambient temperature water is added, and the resulting solution is extracted with ethyl acetate. The combined organic extracts are washed with 1 M hydrochloric acid, water and brine, and dried over magnesium sulfate. After removal of the solvent in vacuo, the residue is purified by chromatography on silica gel (dichloromethane/methanol 20:1->4:1).

Yield: 0.12 g (46% of theory) Mass spectrum (ESI⁺): m/z=610/612 (Cl) [M+H]⁺

Preparation of the end compounds:

### EXAMPLE 1

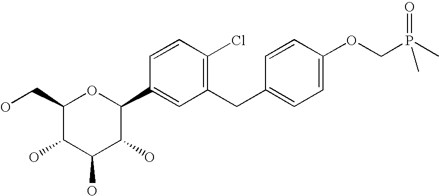

### 1 -Chloro-4-(β-D-glucopyranos-1-yl)-2-[4-(dimethylphosphinoylmethoxy)-benzyl]-benzene

To a solution of 0.65 g 1-chloro4-(2,3,4,6-tetra-O-acetyl-β-D-glucopyranos-1-yl)-2-[4-(dimethylphosphinoylmethoxy)-benzyl]-benzene in 5 mL methanol is added 1.1 mL of 4 M potassium hydroxide solution. After stirring for 1 h at room temperature, the reaction solution is neutralized with 1 M hydrochloric acid, and the solvent is removed in vacuo. The residue was purified by chromatography on reversed phase to afford the product as a white foam.

Yield: 319 mg (67% of theory) Mass spectrum (ESI⁺): m/z=471/473 (Cl) [M+H]⁺

The following compound may be obtained analogously to Example 1:

(2)1 -Chloro-4-(β-D-glucopyranos-1-yl)-2-[4-(dimethylphosphinoyl)-benzyl]-benzene

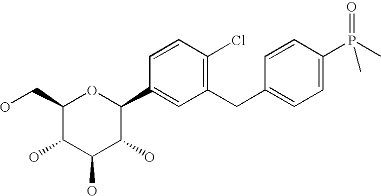

Mass spectrum (ESI⁺): m/z=441/443 (Cl) [M+H]⁺

The following compounds are also prepared analogously to the above-mentioned Examples and other methods known from the literature:

| Ex. | Structure |
|---|---|
| 3 | 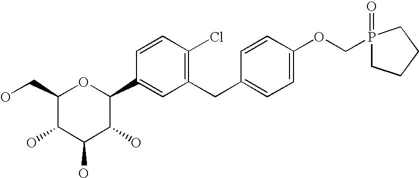 |
| 4 | 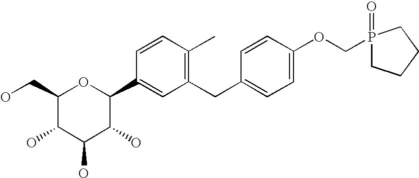 |
| 4 | 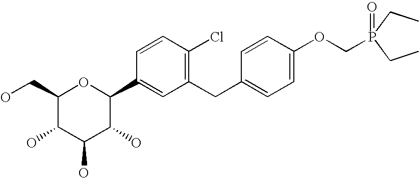 |
| 5 | 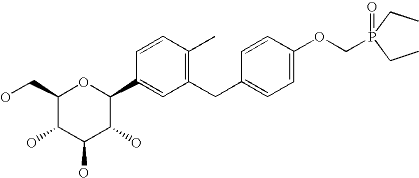 |
| 6 | 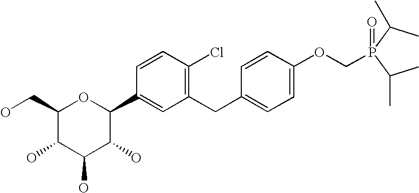 |
| 7 | 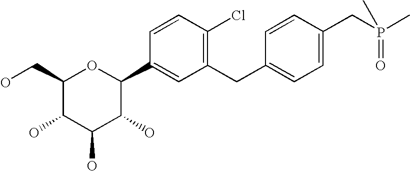 |
| 8 | 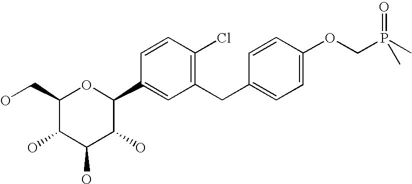 |
| 9 | 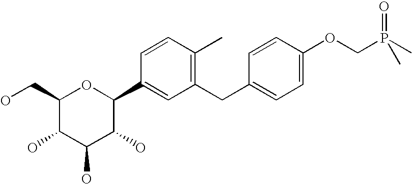 |
| 10 | 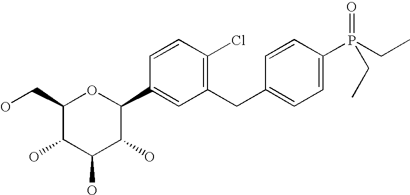 |
| 11 | 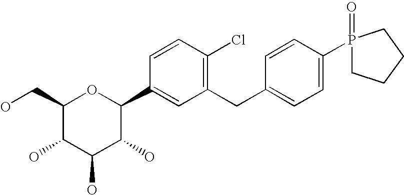 |
| 12 | 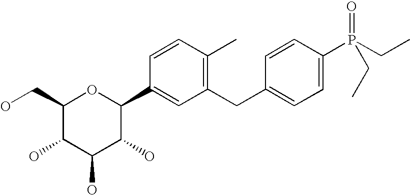 |
| 13 | 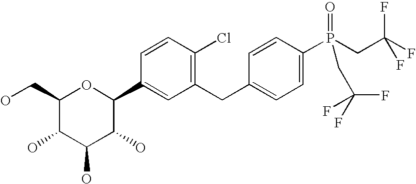 |
| 14 | 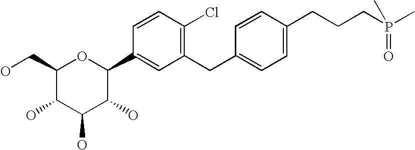 |
| 15 | 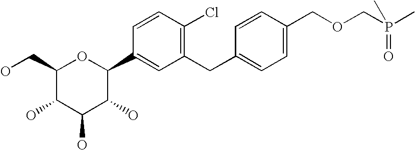 |
| 16 | 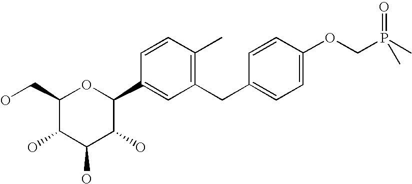 |
| 17 | 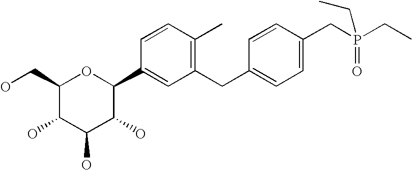 |
| 18 | 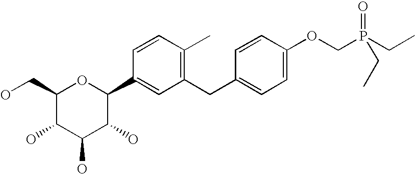 |
| 19 | 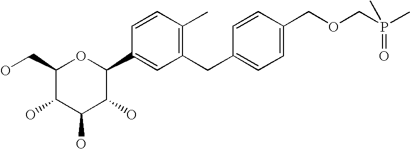 |

Some examples of formulations will now be described in which the term “active substance” denotes one or more compounds according to the invention, including the salts thereof. In the case of one of the combinations with one or additional active substances as described previously, the term “active substance” also includes the additional active substances.

### EXAMPLE A

Tablets containing 100 mg of active substance

Composition:

1 tablet contains:

| | | |
|---|---|---|
| active substance | 100.0 | mg |
| lactose | 80.0 | mg |
| corn starch | 34.0 | mg |
| polyvinylpyrrolidone | 4.0 | mg |
| magnesium stearate | 2.0 | mg |
| | 220.0 | mg |

Method of Preparation:

The active substance, lactose and starch are mixed together and uniformLy moistened with an aqueous solution of the polyvinylpyrrolidone. After the moist composition has been screened (2.0 mm mesh size) and dried in a rack-type drier at 50° C. it is screened again (1.5 mm mesh size) and the lubricant is added. The finished mixture is compressed to form tablets.

| | |
|---|---|
| Weight of tablet: | 220 mg |
| Diameter: | 10 mm, biplanar, facetted on both sides and |
| | notched on one side. |

### EXAMPLE B

Tablets containing 150 mg of active substance

Composition:

1 tablet contains:

| | | |
|---|---|---|
| active substance | 150.0 | mg |
| powdered lactose | 89.0 | mg |
| corn starch | 40.0 | mg |
| colloidal silica | 10.0 | mg |
| polyvinylpyrrolidone | 10.0 | mg |
| magnesium stearate | 1.0 | mg |
| | 300.0 | mg |

Preparation:

The active substance mixed with lactose, corn starch and silica is moistened with a 20% aqueous polyvinylpyrrolidone solution and passed through a screen with a mesh size of 1.5 mm. The granules, dried at 45° C., are passed through the same screen again and mixed with the specified amount of magnesium stearate. Tablets are pressed from the mixture.

| | |
|---|---|
| Weight of tablet: | 300 mg |
| die: | 10 mm, flat |

### EXAMPLE C

Hard gelatine capsules containing 150 mg of active substance

Composition:

1 capsule contains:

| | | | |
|---|---|---|---|
| active substance | | 150.0 | mg |
| corn starch (dried) | approx. | 180.0 | mg |
| lactose (powdered) | approx. | 87.0 | mg |
| magnesium stearate | | 3.0 | mg |
| | approx. | 420.0 | mg |

Preparation:

The active substance is mixed with the excipients, passed through a screen with a mesh size of 0.75 mm and homogeneously mixed using a suitable apparatus. The finished mixture is packed into size 1 hard gelatine capsules.

| | |
|---|---|
| Capsule filling: | approx. 320 mg |
| Capsule shell: | size 1 hard gelatine capsule. |

### EXAMPLE D

Suppositories containing 150 mg of active substance

Composition:

| 1 suppository contains: | | |
|---|---|---|
| active substance | 150.0 | mg |
| polyethyleneglycol 1500 | 550.0 | mg |
| polyethyleneglycol 6000 | 460.0 | mg |
| polyoxyethylene sorbitan monostearate | 840.0 | mg |
| | 2,000.0 | mg |

Preparation:

After the suppository mass has been melted the active substance is homogeneously distributed therein and the melt is poured into chilled moulds.

### EXAMPLE E

Ampoules containing 10 mg active substance

Composition:

| | | |
|---|---|---|
| active substance | 10.0 | mg |
| 0.01 N hydrochloride acid q.s. | | |
| double-distilled water ad | 2.0 | mL |

Preparation:

The active substance is dissolved in the necessary amount of 0.01 N HCI, made isotonic with common salt, filtered sterile and transferred into 2 mL ampoules.

### EXAMPLE F

Ampoules containing 50 mg of active substance

Composition:

| | | |
|---|---|---|
| active substance | 50.0 | mg |
| 0.01 N hydrochloric acid q.s. | | |
| double-distilled water ad | 10.0 | mL |

Preparation:

The active substance is dissolved in the necessary amount of 0.01 N HCI, made isotonic with common salt, filtered sterile and transferred into 10 mL ampoules.

1. Glucopyranosyloxy-substituted benzyl-benzene compound according to general formula I 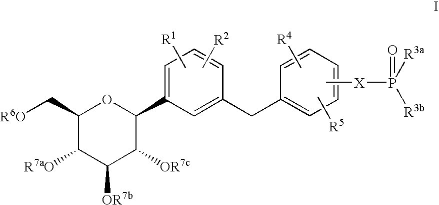
wherein
R¹ denotes hydrogen, fluorine, chlorine, bromine, iodine, C₁₋₄-alkyl, C₂₋₆-alkynyl, C₁₋₄-alkoxy, C₂₋₄-alkenyl-C₁₋₄-alkoxy, C₂₋₄-alkynyl-C₁₋₄-alkoxy, methyl substituted by 1 to 3 fluorine atoms, ethyl substituted by 1 to 5 fluorine atoms, methoxy substituted by 1 to 3 fluorine atoms, ethoxy substituted by 1 to 5 fluorine atoms, C₁₋₄-alkyl substituted by a hydroxy or C₂₋₆-alkoxy group, C₂₋₄-alkoxy substituted by a hydroxy or C₁₋₃-alkoxy group, C₂₋₆-alkenyl, C₃₋₇-cycloalkyl, C₃₋₇-cycloalkyl-C₁₋₃-alkyl, C₃₋₇-cycloalkyl-oxy, C₃₋₇-cycloalkyl-C₁₋₃-alkoxy, C₅₋₇-cycloalkenyloxy, hydroxy, amino, nitro or cyano, while in the C₅₋₆-cycloalkyl groups a methylene group may be replaced by O;
R² denotes hydrogen, fluorine, chlorine, bromine, hydroxy, C₁₋₄-alkyl, C₁₋₄-alkoxy, cyano or nitro, while the alkyl or alkoxy group may be mono- or polysubstituted by fluorine, and
R^{3a}, R^{3b} independently of one another denote C₁₋₆-alkyl, C₂₋₆-alkenyl, C₂₋₆-alkynyl, C₃₋₇-cycloalkyl, C₃₋₇-cycloalkyl-C₁₋₃-alkyl, aryl, heteroaryl, aryl-C₁₋₃-alkyl, heteroaryl-C₁₋₃-alkyl, C₁₋₆alkyloxy, C₄₋₇-cycloalkyloxy, hydroxy;
wherein each C₁₋₅-alkyl group may be substituted with one to three substituents L2; and
wherein aryl-groups may be substituted with one to three substituents L1; or
R3a and R^{3b} are linked together to form a C₄₋₅-alkylene, C4-5-alkenylene, -O-C₃₋₄-alkylene, -O-C₂₋₃-alkylene-O- or -CH₂CH₂-O-CH₂CH₂- chain;
wherein the alkylene moieties may be substituted with one to three substituents L2; and
wherein two adjacent carbon atoms may be part of a further annelated 5- or 6-membered saturated or partially or fully unsaturated carbocyclic ring that may be additionally substituted with up to four substituents L1; and
x denotes bond, 0, C₁₋₅-alkylene, -O-CH₂CH₂-O-, -O-CH₂CH₂-O-CH₂-, -CH₂-O-CH₂CH₂-O-, or an C₂₋₅-alkylene wherein one methylene unit is replaced by O;
wherein the alkylene moieties may be substituted with one to three substituents L2,
R⁴, R⁵ independently of one another denote hydrogen, fluorine, chlorine, bromine, iodine, cyano, nitro, C₁₋₃-alkyl, C₁₋₃-alkoxy, or a methyl- or methoxy-group substituted by 1 to 3 fluorine atoms,
L1 independently of one another are selected from among fluorine, chlorine, bromine, iodine, hydroxy, cyano, C₁₋₃-alkyl, difluoromethyl, trifluoromethyl, C₁₋₃-alkoxy, difluoromethoxy, trifluoromethoxy, amino, C₁₋₃-alkyl-amino and di(C₁₋₃-alkyl)-amino; and
L2 independently of one another are selected from among fluorine, hydroxyl, C₁₋₃-alkyl, difluoromethyl, trifluoromethyl, C₁₋₃-alkoxy, difluoromethoxy, trifluoromethoxy, cyano, amino, C₁₋₃-alkyl-amino and di(C₁₋₃-alkyl)-amino; and
R⁶, R^{7a},
R^{7b}, R^{7c} independently of one another have a meaning selected from among hydrogen, (C₁₋₁₈-alkyl)carbonyl, (C₁₋₁₈-alkyl)oxycarbonyl, arylcarbonyl and aryl-(C₁₋₃-alkyl)-carbonyl, while the aryl-groups may be mono- or disubstituted independently of one another by identical or different groups L1;
while by the aryl groups mentioned in the definition of the above groups are meant phenyl or naphthyl groups which may be substituted as defined; and
while by the heteroaryl groups mentioned in the definition of the above groups are meant a pyrrolyl, furanyl, thienyl, pyridyl, indolyl, benzofuranyl, benzothiophenyl, quinolinyl, isoquinolinyl or tetrazolyl group,
or is meant a pyrrolyl, furanyl, thienyl or pyridyl group, wherein one or two methyne groups are replaced by nitrogen atoms,
or is meant an indolyl, benzofuranyl, benzothiophenyl, quinolinyl or isoquinolinyl group, wherein one to three methyne groups are replaced by nitrogen atoms,
while the above-mentioned heteroaryl groups independently of one another may be mono- or disubstituted by identical or different groups L1;
while, unless otherwise stated, the above-mentioned alkyl groups may be straight or branched chain,
the tautomers, the stereoisomers thereof, the mixtures thereof and the salts thereof.

2. A glucopyranosyloxy-substituted benzyl-benzene compound according to general formula I.2 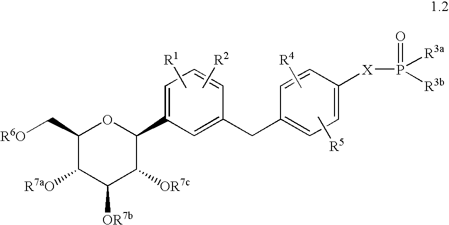
wherein the groups R¹, R², R^{3a}, R^{3b}, R⁴, R⁵, R⁶ and R^{7a}, R^{7b} and R^{7c} are defined as in claim 1.

3. A glucopyranosyloxy-substituted benzyl-benzene derivative according to claim 1 characterised in that the groups R^{3a}, R^{3b} denote independently of one another C₁₋₄-alkyl, phenyl, and C3-7-cycloalkyl, wherein the alkylene parts may be substituted with one to three substituents L2, or
the groups R^{3a}, R^{3b} are linked together to form a C₄₋₅-alkylene chain wherein the alkylene chain may be substituted with one to three substituents L2;
wherein L2 is defined as in claim 1.

4. A glucopyranosyloxy-substituted benzyl-benzene compound according to claim 1, characterised in that the group R¹ denotes hydrogen, fluorine, chlorine, bromine, C₁₋₄-alkyl, C₁₋₄-alkoxy, methyl substituted by 1 to 3 fluorine atoms, methoxy substituted by 1 to 3 fluorine atoms, C₃₋₇-cycloalkyloxy or C₃₋₇-cycloalkyl-C₁₋₃-alkoxy, while in the C₅₋₆-cycloalkyl groups a methylene group may be replaced by O.

5. A glucopyranosyloxy-substituted benzyl-benzene compound according to claim 1, characterised in that the group R² denotes hydrogen, fluorine, chlorine, methyl, methoxy, ethoxy and methyl substituted by 1 to 3 fluorine atoms.

6. A glucopyranosyloxy-substituted benzyl-benzene derivatives according to claim 1, characterised in that the groups R⁴ and/or R⁵ independently of one another represent hydrogen or fluorine.

7. A glucopyranosyloxy-substituted benzyl-benzene compound according to claim 1, characterised in that the group R⁶ denotes hydrogen, (C₁₋₈-alkyl)oxycarbonyl, C₁₋₈-alkylcarbonyl or benzoyl, preferably hydrogen.

8. Glucopyranosyloxy-substituted benzyl-benzene compound according to claim 1, characterised in that the groups R^{7a}, R^{7b}, R^{7c} represent hydrogen.

9. Physiologically acceptable salts of a compound according to claim 1 with inorganic or organic acids.

10. A pharmaceutical composition comprised of a compound according to claim 1 or a physiologically acceptable salt thereof, optionally together with one or more inert carriers and/or diluents.

11. A method of treating diseases or conditions which can be influenced by inhibiting the sodium-dependent glucose cotransporter SGLT, said method comprised of the steps of administering to a patient in need thereof a therapeutically effective amount of a compound according to claim 1 or a physiologically acceptable salt thereof.

12. A method of treating metabolic disorders, said method comprised of the steps of administering to a patient in need thereof a therapeutically effective amount of a compound according to claim 1 or a physiologically acceptable salt thereof.

13. The method of claim 12 wherein the metabolic disorder is selected from the group consisting of type 1 and type 2 diabetes mellitus, complications of diabetes, metabolic acidosis or ketosis, reactive hypoglycaemia, hyperinsulinaemia, glucose metabolic disorder, insulin resistance, metabolic syndrome, dyslipidaemias of different origins, atherosclerosis and related diseases, obesity, high blood pressure, chronic heart failure, edema and hyperuricaemia.

14. A method of treating diseases or conditions which can be influenced by inhibiting the sodium-dependent glucose cotransporter SGLT, said method comprised of the steps of administering to a patient in need thereof a therapeutically effective amount of a pharmaceutical composition comprised of a compound according to claim 1 or a physiologically acceptable salt thereof.

15. A method of preventing the degeneration of pancreatic beta cells and/or for improving and/or restoring the functionality of pancreatic beta cells, said method comprised of the steps of administering to a patient in need thereof a therapeutically effective amount of a compound according to claim 1 or a physiologically acceptable salt thereof.

16. A method of treating conditions requiring the use of diuretics or antihypertensives, said method comprised of the steps of administering to a patient in need thereof a therapeutically effective amount of a compound according to claim 1 or a physiologically acceptable salt thereof.

17. A process for preparing a pharmaceutical composition comprised of a compound according claim 1 or a physiologically acceptable salt thereof, said method comprised of the incorporating said compound into one or more inert carriers and/or diluents by a non-chemical method.

18. Compounds of general formula IVa and IVb 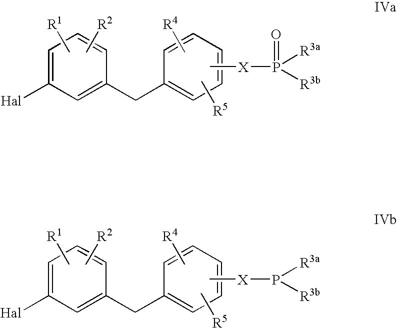
wherein Hal denotes chlorine, bromine or iodine and the groups R¹, R², R^{3a}, R^{3b}, R⁴ and R⁵ are defined as in claim 1.
